(19) United States
(12) Patent Application Publication
(10) Pub. No.: US 2026/0126382 A1
(43) Pub. Date: May 7, 2026

(54) INTELLIGENT NIR II PHOTOTHERMAL AND COLORIMETRIC SENSING TECHNOLOGY

(71) Applicant: Nanyang Technological University, Singapore (SG)

(72) Inventors: Bengang XING, Singapore (SG); Ling QIAO, Singapore (SG); Wenchao LANG, Singapore (SG)

(21) Appl. No.: 19/117,534

(22) PCT Filed: Oct. 27, 2023
(86) PCT No.: PCT/SG2023/050719

§ 371 Date: Apr. 1, 2025

(30) Foreign Application Priority Data
Oct. 27, 2022 (SG) 10202251512P

(51) Int. Cl. G01N 21/359; C12Q 1/34; G01N 33/00; G01N 33/573

(52) U.S. Cl. CPC G01N 21/359; C12Q 1/34; G01N 33/0098; G01N 33/573; G01N 2333/924

(57) ABSTRACT

The current invention relates to a method of quantitatively determining the concentration of active myrosinase in a sample, the method comprising the steps of (a) adding a portion of the sample comprising active myrosinase to a first analyte assay formulation for a first period of time to generate an analyte sample, (b) adding a portion of the sample comprising inactivated myrosinase to a second analyte assay formulation for a second period of time to generate a analyte reference sample, and (c) determining the concentration of the active myrosinase in the sample. In an embodiment, the first anayte assasy formulation comprising a near infra-red absorbing and photothermally-responsive compound. The invention also relates to a formulation for use in a method of quantitatively determining the concentration of active myrosinase in a sample, and a kit of parts suitable to provide a formulation as aforementioned.

### FIELD OF INVENTION

The present disclosure relates to methods of sensing of myrosinase, and more particularly relates to methods of quantitatively determining the concentration of active myrosinase in a sample.

### BACKGROUND

The listing or discussion of a prior-published document in this specification should not necessarily be taken as an acknowledgement that the document is part of the state of the art or is common general knowledge.

Myrosinase (Myr), a member of the glycoside hydrolase family produced in commonly consumed cruciferous vegetables such as broccoli, cauliflower, watercress, Brussels sprouts, and cabbage, can catalyze the hydrolysis of inactive glucosinolates (GLs) into various compounds. Among the hydrolyzed product, isothiocyanates (ITCs) possess powerful chemopreventive effects, including anti-inflammatory, antioxidant, and antitumor effects. Studies have shown that increased ingestion of cruciferous vegetables can reduce the incidence of various diseases, which reveal the chemopreventive effects of GLs-Myr-ITCs system from dietary intake. However, Myr is significantly inactivated through vegetable storage, transporting and cooking process. Owing to merits of diet therapy and vulnerability of enzyme, the detection and profiling of Myr to better utilize chemoprevention through daily consumption are significantly important.

The analysis of Myr by measuring the substrate (GLs) consumption or specific product generation such as glucose (GO) have been reported. The classical techniques mainly include spectrophotometric assay (UV), high-performance liquid chromatography (HPLC), gas chromatography-mass spectrometry (GC-MS), and pH-salt assay. However, these methods also have some limitations in practical applications owing to their complex, time-consuming protein purification steps. There are also some revised methods for detecting Myr, such as immunological methods and on-gel detection, to avoid tedious purification steps. Nonetheless, some drawbacks persist, such as immunological methods that may not be capable of recognizing denatured Myr with no nutritive value, and on-gel detection which requires extensive gel preparations, which is in turn time-consuming, labor-intensive, and not suitable for onsite Myr testing. As such, despite the promising nutrition-activating potential of Myr derived from cruciferous vegetables, the monitoring and profiling of Myr in food inspection and nutrition evaluation is restricted due to the lack of suitable analysis methods. A simple, fast, effective, visualized myrosinase profiling method is still unavailable.

Therefore, there exists a need to develop a sensing technology with intelligent display and comparison for onsite screening in food industry.

### SUMMARY OF INVENTION

It has been surprisingly found that a simple method can be used to determine the level of myrosinase in a sample-whether from a vegetable or from a dietary supplement or other source. Aspects and embodiments of the invention are provided in the following numbered clauses.
1. A method of quantitatively determining the concentration of active myrosinase in a sample, the method comprising the steps of:
(a) adding a portion of the sample comprising active myrosinase to a first analyte assay formulation for a first period of time to generate an analyte sample, the first analyte assay formulation comprising:
   a substrate for active myrosinase that provides glucose as a reaction product;
   a glucose oxidase;
   either a near infra-red absorbing and photothermally-responsive compound capable of forming a charge transfer complex or nanoparticle aggregates; and
   either a peroxidase or nanoparticles having peroxidase-like activity;
(b) adding a portion of the sample comprising inactivated myrosinase to a second analyte assay formulation for a second period of time to generate a analyte reference sample, the second analyte assay formulation being the same as the first analyte assay formulation and the second period of time being the same as the first period of time;
(c) determining the concentration of the active myrosinase in the sample by measuring one or more of:
   (i) Red Green Blue colour values in the analyte sample and Red Green Blue colour values in the analyte reference sample, calculating the Red/Blue ratio for each of the analyte sample and the analyte reference sample and calculating the difference between the Red/Blue ratios to provide a sample Red/Blue ratio and comparing the sample Red/Blue ratio obtained to a pre-determined calibration curve of active myrosinase concentration based on Red/Blue ratios obtained from known concentrations of active myrosinase;
   (ii) an infra-red image of the analyte sample and an infra-red image of the analyte reference sample obtained following irradiation of the analyte sample and the analyte reference same by a laser for a third period of time and calculating the temperature difference between a temperature obtained from the infra-red image of the analyte sample and a temperature obtained from the infra-red image of the analyte reference sample and comparing the difference value obtained to a pre-determined calibration curve of active myrosinase concentration based on temperatures obtained from infra-red images of known concentrations of active myrosinase;
   (iii) an absorbance spectra of the analyte sample and an absorbance spectra of the analyte reference sample and calculating the difference between the absorbance spectra of the analyte sample and the absorbance spectra of the analyte reference sample and comparing the difference value obtained to a pre-determined calibration curve of active myrosinase concentration based on absorbance spectra of known concentrations of active myrosinase; and
   (iv) a temperature signal obtained following irradiation of the analyte sample and the analyte reference sample by a laser for a third period of time and calculating the difference between the temperature signal of the analyte sample and the temperature signal of the analyte reference sample and comparing the difference value obtained to a pre-determined calibration curve of active myrosinase concentration based on temperature signals of known concentrations of active myrosinase.
2. The method according to Clause 1, wherein two or more of (i) to (iv) are used to determine the concentration of active myrosinase in the sample.
3. The method according to Clause 1 or Clause 2, wherein the substrate for active myrosinase that provides glucose as a reaction product is a glucosinolate, optionally wherein the glucosinolate is selected from the group consisting of glucotropaeolin, gluconasturtiin, glucoraphanin, and sinigrin.
4. The method according to any one of the preceding claims, wherein the near infra-red absorbing and photothermally-responsive compound capable of forming a charge transfer complex or nanoparticle aggregates is selected from phenylboronate decorated gold nanoparticles, or one or more of the group selected from perylene, F4TCNQ, tetracyanoquinodimethane (TCNQ), and 3,3′,5,5′-tetramethylbenzidine, optionally wherein the near infra-red absorbing and photothermally-responsive compound capable of forming a charge transfer complex or nanoparticle aggregates is 3,3′,5,5′-tetramethylbenzidine.
5. The method according to any one of the preceding clauses, wherein the nanoparticles having peroxidase-like activity are selected from silver or, more particularly, gold nanoparticles, optionally wherein the gold nanoparticles have one or more of the following properties:
(ai) an absorption peak at about 527 nm;
(aii) an average diameter size of about 13 nm as determined from transmission electron microscopy;
(aiii) a zeta potential in an aqueous solution of from +20 to +30 mV, such as about +24.5 mV; and
(aiv) infra-red absorption peaks at about 1650 cm⁻¹ and about 3450 cm⁻¹.
6. The method according to any one of the preceding clauses, wherein one or more of the following apply:
(bi) when the nanoparticles having peroxidase-like activity or the peroxidase are gold nanoparticles, they are present in the first and second analyte assay formulations at a concentration of from 0.02 to 0.8 nM, such as about 0.45 nM;
(bii) when the near infra-red absorbing and photothermally-responsive compound capable of forming a charge transfer complex or nanoparticle aggregates is 3,3′,5,5′-tetramethylbenzidine, then it is present in the first and second analyte assay formulations at a concentration of from 0.5 to 3 mM, such as about 1.5 mM;
(biii) the glucose oxidase is present in an amount of from 5 U/mL to 25 U/mL;
(biv) the substrate for active myrosinase that provides glucose as a reaction product is present in an amount of from 0.05 mM to 0.75 mM.
7. The method according to any one of the preceding clauses, wherein the first and second analyte assay formulations further comprise an acetate buffer in an amount that provides a pH of from 3.5 to 7.5, such as about 4.5.
8. The method according to any one of the preceding clauses, wherein the sample comprising active myrosinase and the sample comprising inactivated myrosinase is provided at a concentration of from 1 to 20 mg/mL, such as about 10 mg/mL.
9. The method according to any one of the preceding clauses, wherein the sample comprising inactivated myrosinase is obtained by heating a sample comprising active myrosinase to a temperature suitable to denature active myrosinase for a fourth period of time, optionally wherein one or both of the following apply:
   the temperature is from 80 to 120° C., such as about 100° C.; and
   the fourth period of time is from 30 minutes to 2 hours, such as about 1 hour.
10. The method according to any one of the preceding clauses, wherein the sample comprising active myrosinase is subjected to incubation for a fifth period of time at a suitable temperature before use in the method, optionally wherein one or both of the following apply:
   the temperature is from 15 to 30° C., such as about 25° C.; and
   the fifth period of time is from 10 minutes to 1 hour, such as about 30 minutes.
11. The method according to any one of the preceding clauses, wherein when one or both of (ii) and (iv) of step (c) in Clause 1 are used to determine the concentration of the active myrosinase in the sample, then the laser is a near infra-red light laser, optionally wherein one or more of the following apply:
   the laser provides a light beam having a wavelength from 1000 to 1500 nm, such as about 1064 nm;
   the laser has a power density of from 0.5 to 3 W/cm², such as about 1 W/cm²; and
the third period of time is from 20 seconds to 5 minutes.
12. The method according to any one of the preceding clauses, wherein the first and second periods of time are from 10 minutes to 1 hour, such as 30 minutes.
13. The method according to any one of the preceding clauses, wherein the absorbance spectra of the analyte sample and the analyte reference sample are based on their absorbance at a specified wavelength or a wavelength range in the near infra-red range, optionally wherein the specified wavelength is a wavelength selected from 1,000 to 1,500 nm, such as 1064 nm.
14. The method according to any one of the preceding clauses, wherein the sample is obtained from a dietary supplement comprising myrosinase, wasabi (e.g. wasabi powder), or a cruciferous plant, optionally wherein the cruciferous plant is selected from a broccoli, a cauliflower, a cabbage, and a Chinese cabbage.
15. The method according to any one of the preceding clauses, wherein the first and second analyte assay formulations comprise:
   sinigrin;
   a glucose oxidase;
   3,3′,5,5′-tetramethylbenzidine; and
   gold nanoparticles.
16. The method according to any one of the preceding clauses, wherein the method further comprises a step (d), which is selected from one or more of the following:
(di) retaining or disposing of a batch from which the sample was drawn based on a level of freshness of the batch based on the concentration of the active myrosinase in the sample; and
(dii) labelling a batch from which the sample was drawn with a specific level or amount of active myrosinase.
17. A formulation for use in a method of quantitatively determining the concentration of active myrosinase in a sample, the formulation comprising:
   a substrate for active myrosinase that provides glucose as a reaction product;
   a glucose oxidase;
   either a near infra-red absorbing and photothermally-responsive compound capable of forming a charge transfer complex or nanoparticle aggregates; and
      either a peroxidase or nanoparticles having peroxidase-like activity.
18. The formulation according to Clause 17, wherein the substrate for active myrosinase that provides glucose as a reaction product is a glucosinolate, optionally wherein the glucosinolate is selected from the group consisting of glucotropaeolin, gluconasturtiin, glucoraphanin, and sinigrin.
19. The formulation according to Clause 17 or Clause 18, wherein the near infra-red absorbing and photothermally-responsive compound capable of forming a charge transfer complex or nanoparticle aggregates is selected from phenylboronate decorated gold nanoparticles, or one or more of the group selected from perylene, F4TCNQ, tetracyanoquinodimethane (TCNQ), and 3,3′,5,5′-tetramethylbenzidine, optionally wherein the near infra-red absorbing and photothermally-responsive compound capable of forming a charge transfer complex or nanoparticle aggregates is 3,3′,5,5′-tetramethylbenzidine.
20. The formulation according to any one of Clauses 17 to 19, wherein the formulation comprises:
   sinigrin;
   a glucose oxidase;
   3,3′,5,5′-tetramethylbenzidine; and
   gold nanoparticles.
21. The formulation according to any one of Clauses 17 to 20, wherein the formulation further comprises an acetate buffer capable of generating an aqueous solution that has a pH of from 3.5 to 7.5, such as about 4.5.
22. A kit of parts suitable to provide a formulation according to any one of Clauses 17 to 21, wherein the kit of parts comprises:
   a substrate for active myrosinase that provides glucose as a reaction product, optionally wherein the substrate for active myrosinase that provides glucose as a reaction product is sinigrin;
   a glucose oxidase;
   either a near infra-red absorbing and photothermally-responsive compound capable of forming a charge transfer complex or nanoparticle aggregates; and
   either a peroxidase or nanoparticles having peroxidase-like activity.
23. The kit of parts according to Clause 22, wherein the kit of parts comprises:
   sinigrin;
   a glucose oxidase;
   3,3′,5,5′-tetramethylbenzidine; and
   gold nanoparticles.
24. The kit of parts according to Clause 22 or Clause 23, wherein the kit of parts further comprises an acetate buffer capable of generating an aqueous solution that has a pH of from 3.5 to 7.5, such as about 4.5.

### DRAWINGS

FIG. 1 is a schematic illustration of rapid and simple NIR-II photothermal responsive onsite Myr detection via smartphone readout.

FIG. 2 depicts optimization of preparation time for AuNPs. (A) Absorption of the system (TMB+H₂O₂+AuNPs) under different AuNPs preparation time, and (B) absorbance at 1064 nm vs. preparation time of AuNPs.

FIG. 3 depicts (A) size distribution of synthesized gold nanoparticles (AuNPs), the top right inset is the transmission electron microscopy (TEM) image of AuNPs, (B) Ultraviolet-visible-Near Infrared (UV-vis-NIR) spectrum and colorimetric images for H₂O₂ detection, (C) Abs1064 nm under different pH values. The lower left inset is the TEM image of charge transfer complex (CTC) at pH 4.5, and the upper right inset is the TEM image at pH 8.0, and (D) temperature (ΔTemp) signals and infrared thermal images for H₂O₂ detection, the power density of the 1064 nm laser is 1 W/cm²: (i) only 3,3′,5,5′-tetramethylbenzidine (TMB); (ii) TMB+AuNPs; (iii) TMB+H₂O₂; (iv) H₂O₂+AuNPs; and (v) TMB+H₂O₂+AuNPs; the final concentrations of H₂O₂, TMB, and AuNPs were 0.25 mM, 1.5 mM and 0.45 nM, respectively.

FIG. 4 depicts an absorption spectrum of the obtained AuNPs.

FIG. 5 depicts (A) Fourier transform infrared (FTIR) spectrum of the obtained AuNPs, and (B) Zeta potential of the obtained AuNPs.

FIG. 6 depicts (A) formation of CTC, and (B) absorption spectrum of TMB+H₂O₂+AuNPs system under different pH values.

FIG. 7 depicts optimization of AuNPs concentration. (A) Absorption spectrum of the system (TMB+H₂O₂+AuNPs) under different AuNPs concentration, and (B) absorbance at 1064 nm vs. c (AuNPs). The upper left inset is the colorimetric image.

FIG. 8 depicts optimization of TMB concentration. (A) UV-vis-NIR spectrum, and (B) absorbance at 1064 nm under different TMB concentration.

FIG. 9 depicts optimization of working temperature. (A) Effect of temperature on UV-vis-NIR spectrum, and (B) Abs1064 nm vs. temperature.

FIG. 10 depicts (A) absorption spectrum of the TMB+H₂O₂+AuNPs system in the presence of different concentrations of H₂O₂, and (B) relationship between Abs1064 nm and c (H₂O₂); inset shows the linear calibration plot for H₂O₂ detection.

FIG. 11 depicts (A) absorbance spectra and (B) Abs1064 nm vs. c(HRP) of TMB+H₂O₂ system. Inset is linear relationship between Abs1064 nm and c(HRP).

FIG. 12 depicts optimization of working conditions of TMB concentration, pH, and temperature. (A) UV-Vis-NIR spectrum and (B) absorbance at 1064 nm under different TMB concentration; (C) Absorption spectrum and (D) Abs1064 nm under different pH values. The upper right inset is TEM image of system (TMB+H₂O₂+AuNPs) at pH 4.5, and the lower left inset is TEM image at pH 8.0. (E) Effect of temperature on UV-Vis-NIR spectrum and (F) Abs1064 nm vs. temperature.

FIG. 13 depicts (A) UV-Vis-NIR spectra and colorimetric images and (B) ΔTemp signals and infrared thermal images for Myr detection; (i) Sin+Myr+GOx+TMB, (ii) Sin+Myr TMB+AuNPs, (iii) Sin+GOx+TMB+AuNPs, (iv) Myr+GOx+TMB+AuNPs, (v) Sin+Myr+GOx+AuNPs, and (vi) with Sin+Myr+GOx+TMB+AuNPs. The final concentrations of Sin, Myr, GOx, TMB, and AuNPs were 0.75 mM, 0.75 mg/ml, 0.5 mg/ml, 1.5 mM, and 0.45 nM, respectively. The power density of the 1064 nm laser was 1 W/cm², (C) absorbance at 1064 nm of the Myr test system with Cu²⁺ and ethylenediaminetetraacetic acid (EDTA); inset shows the colorimetric images, and (D) ΔTemp signals after being irradiated by a 1064 nm laser at 1 W/cm2 for 5 min; inset is the infrared thermal images.

FIG. 14 depicts absorption spectra of Myr test system with Cu²⁺ and EDTA.

FIG. 15 depicts (A) UV-Vis-NIR spectrum and colorimetric images and (B) temperature signals (ΔTemp) and infrared thermal images for GO detection (i) GO+GOx+TMB, (ii) GO+TMB+AuNPs, (iii) GO+GOx+TMB+AuNPs, and the concentration of GO, GOx, TMB, and AuNPs were 0.45 mM, 0.5 mg/mL, 1.5 mM, and 0.45 nM, respectively.

FIG. 16 depicts (A) absorption spectrum of Sin+Myr+GOx+TMB+AuNPs system with different concentrations of Sin; the inset at top right is the corresponding colorimetric image, and (B) relationship between Abs1064 nm and c (Sin), the final concentration of Myr, GOx, TMB, and AuNPs were 0.5 mg/mL, 0.5 mg/mL, 1.5 mM, and 0.45 nM, respectively.

FIG. 17 depicts (A) absorption spectrum of different concentrations of Myr; the corresponding colorimetric image is exhibited in the top right, (B) linear relationship between Abs1064 nm and Myr enzyme within the range of 0-172.5 mU/mL (i), and relationship between temperature and logarithm of Myr enzyme concentration (ii), (C) absorbance at 1064 nm of Myr and various interfering substances; inset shows the colorimetric images, and (D) ΔTemp signals after being irradiated by a 1064 nm laser at 1 W/cm² for 5 min, and infrared thermal images

FIG. 18 depicts the plot of Abs1064 nm and c (Myr). The fitted linear equation was Abs_{1064 nm}=4.39×10⁻⁴×Myr (mU/mL)+0.0298, R²=0.99616, and limit of detection (LOD) was estimated to be 2.96 mU/mL according to the 30/s, where σ represents the standard deviation obtained by blank samples and s is the slope of the calibration curve.

FIG. 19 depicts (A) absorption spectrum of the TMB+H₂O₂+AuNPs system in the presence of different concentrations of H₂O₂, and (B) relationship between Abs1064 nm and c (H₂O₂); inset shows the linear calibration plot for H₂O₂ detection.

FIG. 20 depicts (A) absorption spectrum of the GO+GOx+TMB+AuNPs system with increasing c(GO), and (B) relationship between Abs1064 nm and c(GO); inset shows the linear calibration plot for GO detection.

FIG. 21 depicts the effects of amino acids (1 mM), small molecules (1 mM), and metal ions (1 mM) on Myr detection; c (Myr) was 0.75 mg/mL.

FIG. 22 depicts effects of amino acids (1 mM), small molecules (1 mM), and metal ions (1 mM) on Myr detection, c (Myr) is 0.5 mg/mL.

FIG. 23 depicts (A) temperature responses with increasing concentrations of Myr within 5 min and (B) the corresponding infrared thermal image at a different time under 1064 nm laser irradiation with a power of 1 W/cm², and (C) thermal cycle stability of the Sin+Myr+GOx+TMB+AuNPs system.

FIG. 24 depicts (A) the plot of B/R vs. c (Myr) in the range of 0-345 mU/mL, (B) presents a linear relationship of B/R values with Myr concentrations from 0 to 86.25 mU/mL, and (C) the linear calibration plot of B/R value versus the concentration of Myr in the range of 86.25-345 mU/mL.

FIG. 25 depicts (A) ΔAbs spectrum and (B) temperature responses of various vegetables during 5 min. Carrot, lettuce, Xiao Bai Cai, Chinese cabbage, cauliflower, green cabbage, and broccoli are represented by the numerals i, ii, iii, iv, v, vi, and vii, respectively; the concentration of all the vegetables was 10 mg/mL.

FIG. 26 depicts (A) ΔAbs of some vegetables at 1064 nm; inset shows the colorimetric images, (B) temperature of several vegetables after being irradiated by a 1064 nm laser at 1 W/cm² for 5 min; inset is the infrared thermal images, (C) diagram of Myr enzyme determination by Calculator, and (D) B/R value and Myr concentration in actual vegetable samples after being processed by Calculator. Carrot, lettuce, Xiao Bai Cai, Chinese cabbage, cauliflower, green cabbage, and broccoli are represented by the numerals i, ii, iii, iv, v, vi, and vii, respectively.

FIG. 27 depicts (A) absorbance spectrum and (B) Abs1064 nm and temperature change for 5 mg/mL of broccoli and cabbage. The numbers i and ii represent broccoli and cabbage, respectively.

FIG. 28 depicts (A) Δabsorbance at 1064 nm of some types of vegetables; inset shows the colorimetric images, and (B) ΔTemp of several vegetables after being irradiated by 1064 nm laser at 1 W/cm² for 5 mins, inset is the infrared thermal images. Carrot, lettuce, Xiao Bai Cai, Chinese cabbage, cauliflower, green cabbage, broccoli are represented by the numerals i, ii, iii, iv, v, vi, and vii, respectively.

FIG. 29 depicts a flow chart of image analysis and processing of “Calculator” website.

FIG. 30 depicts screenshots and illustration of the developed “Calculator” interface. (A) Illustration of “Calculator” user interface, (B) screenshot of the initial “Calculator” interface, (C) screenshot of the “Calculator” image acquiring, and (D) screenshot of the “Calculator” data processing.

FIG. 31 depicts (A) the plot of B/R vs. c (Myr) in the range of 0-345 mU/mL, (B) presents a linear relationship between B/R values and Myr concentrations from 0 to 86.25 mU/mL, (C) the linear calibration plot of B/R value versus the concentration of Myr in the range of 86.25-345 mU/mL, and (D) the plot of B/R value vs. c (Myr) in actual vegetable samples after being processed by “Calculator” website. The concentration of all vegetables was 10 mg/mL.

FIG. 32 depicts (A) absorption spectrum and (B) Abs1064 nm and temperature change for wasabi powder (1 mg/mL) and dietary supplement (10 mg/mL). (C) Screenshots of Myr detection by smartphone and corresponding results in (D). The numbers i and ii represent dietary supplement (10 mg/mL) and wasabi powder (10 mg/mL), respectively.

FIG. 33 depicts a universal EV extraction method.

FIG. 34 depicts characterization of obtained EV.

FIG. 35 depicts synthesis of engineered hybrid vesicles containing Myr.

FIG. 36 depicts various Myr substrates for analytic and theranostic application.

FIG. 37 depicts “homing effect” possessed by different sourced EVs for lesion affinity.

FIG. 38 depicts imaging and theranostic evaluation in different levels.

### DESCRIPTION

The current invention relates to a sensing technology that can use one or both of colorimetric and photothermal signals for rapid onsite myrosinase profiling in actual samples precisely using NIR-II absorbance window, which may be done in some instances using a smartphone readout and intelligent data processing on a smartphone-based calculating website. This sensing platform based on colorimetric and temperature response not only provides a new strategy for rapid qualitative and quantitative onsite myrosinase analysis (e.g. via smartphone readout), but also shows potential applications in the fields of food quality analysis and bacteria screening. This screening method does not necessarily need to make use of expensive components, but may also use a set-up involving near infra-red II (NIR-II) lasers, cameras (IR and visible light) and computing systems to effect rapid qualitative and quantitative onsite myrosinase analysis.

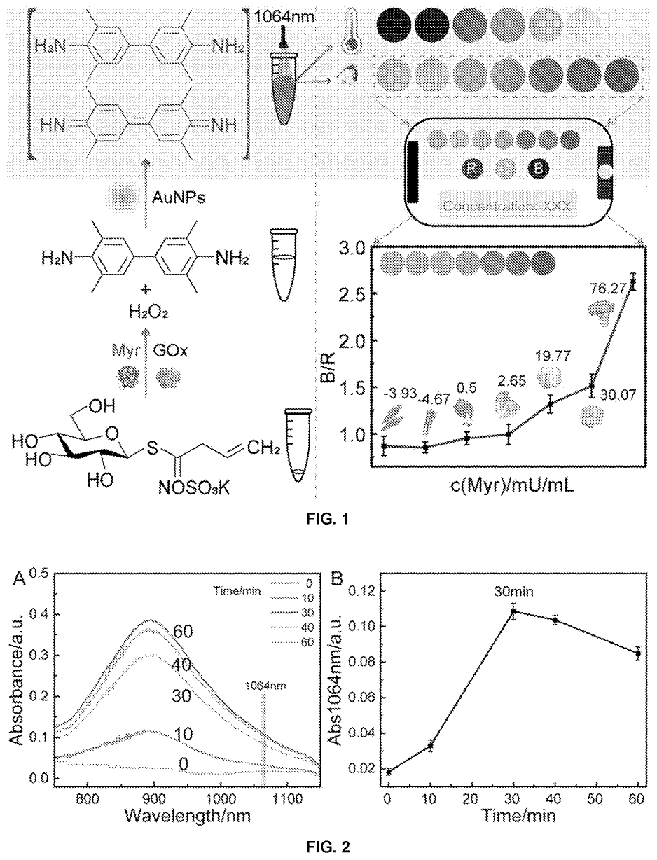

FIG. 1 shows an example of the developed intelligent screening technology for myrosinase profiling from various substrates, such as vegetables, food supplements, and bacteria. Briefly, sinigrin (Sin), myrosinase (Myr) and glucose oxidase (GOx) were first mixed for 30 min to generate H₂O₂, then 3,3′,5,5′-tetramethylbenzidine (TMB) and gold nanoparticles (AuNPs) were added, incubated at 45° C. for 25 min. After cooling to room temperature, the solution changed from colourless to blue due to the formation of a charge transfer complex (CTC), which possessed broad absorption from UV to NIR-II window. Furthermore, the temperature signals and infrared thermal images could be obtained, due to the CTC's photothermal effect after irradiation by a 1064 nm laser. Thus, the combined detection of Myr by colorimetric methods and temperature can be achieved. For the purpose of detecting Myr in actual samples intelligently, a smartphone-based website named “Calculator” can be used to obtain the relevance between the Blue/Red (B/R) values of colorimetric images and Myr concentrations. The Myr concentrations in actual samples can then be determined according to the formula via smartphone readout.

Thus, in a first aspect of the invention, there is provided a method of quantitatively determining the concentration of active myrosinase in a sample, the method comprising the steps of:
(a) adding a portion of the sample comprising active myrosinase to a first analyte assay formulation for a first period of time to generate an analyte sample, the first analyte assay formulation comprising:
   a substrate for active myrosinase that provides glucose as a reaction product;
   a glucose oxidase;
   either a near infra-red absorbing and photothermally-responsive compound capable of forming a charge transfer complex or nanoparticle aggregates; and
   either a peroxidase or nanoparticles having peroxidase-like activity;
(b) adding a portion of the sample comprising inactivated myrosinase to a second analyte assay formulation for a second period of time to generate a analyte reference sample, the second analyte assay formulation being the same as the first analyte assay formulation and the second period of time being the same as the first period of time;
(c) determining the concentration of the active myrosinase in the sample by measuring one or more of:
   (i) Red Green Blue colour values in the analyte sample and Red Green Blue colour values in the analyte reference sample, calculating the Red/Blue ratio for each of the analyte sample and the analyte reference samples and calculating the difference between the Red/Blue ratios to provide a sample Red/Blue ratio and comparing the sample Red/Blue ratio obtained to a pre-determined calibration curve of active myrosinase concentration based on Red/Blue ratios obtained from known concentrations of active myrosinase;
   (ii) an infra-red image of the analyte sample and an infra-red image of the analyte reference sample obtained following irradiation of the analyte sample and the analyte reference same by a laser for a third period of time and calculating the temperature difference between a temperature obtained from the infra-red image of the analyte sample and a temperature obtained from the infra-red image of the analyte reference sample and comparing the difference value obtained to a pre-determined calibration curve of active myrosinase concentration based on temperatures obtained from infra-red images of known concentrations of active myrosinase;
   (iii) an absorbance spectra of the analyte sample and an absorbance spectra of the analyte reference sample and calculating the difference between the absorbance spectra of the analyte sample and the absorbance spectra of the analyte reference sample and comparing the difference value obtained to a pre-determined calibration curve of active myrosinase concentration based on absorbance spectra of known concentrations of active myrosinase; and
   (iv) a temperature signal obtained following irradiation of the analyte sample and the analyte reference sample by a laser for a third period of time and calculating the difference between the temperature signal of the analyte sample and the temperature signal of the analyte reference sample and comparing the difference value obtained to a pre-determined calibration curve of active myrosinase concentration based on temperature signals of known concentrations of active myrosinase.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The sample may be any suitable material that contains, or is purported to contain, active myrosinase. Examples of such materials include, but are not limited to a dietary supplement comprising myrosinase, wasabi (e.g. wasabi powder), or a cruciferous plant. Any suitable cruciferous plant may be tested using the method disclosed herein. For example, the cruciferous plant may be, but is not limited to, a broccoli, a cauliflower, a cabbage, and a Chinese cabbage.

As noted herein, the method makes use of two samples derived from the same source material. One of the samples (i.e. the analyte sample) is used as-is, so as to preserve the activity of the myrosinase present in the sample. The other sample (i.e. the analyte reference sample) is obtained by inactivating the myrosinase through some form of denaturisation. For example, the sample comprising inactivated myrosinase may be obtained by heating a sample comprising active myrosinase to a temperature suitable to denature active myrosinase for a period of time (e.g. from 30 minutes to 2 hours, such as about 1 hour). Suitable temperatures may include, but are not limited to a temperature of from 80 to 120° C., such as about 100° C.

As the selected sample may contain an endogenous amount of glucose, the analyte reference sample is used to determine a “background” level of glucose in the sample, so that this does not affect the determination of the actual amount of myrosinase in the sample.

As noted herein, the method makes use of a first analyte assay formulation and a second analyte assay formulation. These formulations are essentially identical, with the only difference being that one is added to the sample containing active myrosinase, while the other is added to the sample with deactivated myrosinase. Both of these formulations comprise:
a substrate for active myrosinase that provides glucose as a reaction product
a glucose oxidase;
either a near infra-red absorbing and photothermally-responsive compound capable of forming a charge transfer complex or nanoparticle aggregates; and
either a peroxidase or nanoparticles having peroxidase-like activity.

Any substrate that can be provided to active myrosinase to provide glucose may be used herein. For example, the substrate may be a glucosinolate. Any suitable glucosinolate may be used herein. Examples of suitable glucosinolates include, but are not limited to, glucotropaeolin, gluconasturtiin, glucoraphanin, and sinigrin. In particular embodiments of the invention that may be mentioned herein, the glucosinolate may be sinigrin.

The near infra-red absorbing and photothermally-responsive compound capable of forming a charge transfer complex or nanoparticle aggregates may be any suitable material with these properties. Suitable nanoparticle aggregates may be phenylboronate decorated gold nanoparticles. Other suitable nanoparticles may be decorated with thioketals, thioesters or other boronic esters. These species are reactive oxygen species-sensitive and so will be cleaved by a reactive oxygen species (e.g. a peroxide) into monomers, causing the nanoparticle aggregates to lose structural integrity and thereby providing a photothermal response.

Suitable charge transfer complexes may be selected from, but are not limited to the list of perylene, F4TCNQ, tetracyanoquinodimethane (TCNQ), 3,3′,5,5′-tetramethylbenzidine (TMB), and combinations thereof (e.g. TMB-F4TCNQ and TMB-TCNQ). In particular embodiments of the invention, the near infra-red absorbing and photothermally-responsive compound capable of forming a charge transfer complex or nanoparticle aggregates may be 3,3′,5,5′-tetramethylbenzidine.

TCNQ has the structure:

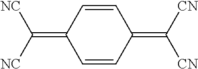 [SMILES: N#CC(C#N)=c1ccc(=C(C#N)C#N)cc1]

F4TCNQ has the structure:

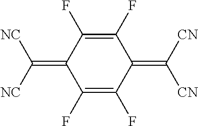 [SMILES: N#CC(C#N)=c1c(F)c(F)c(=C(C#N)C#N)c(F)c1F]

When a peroxidase is present, any suitable peroxidase may be used. An example of a suitable peroxidase is horseradish peroxidase.

When the nanoparticles having peroxidase-like activity are present, these may be silver or, more particularly, gold nanoparticles. Such silver and gold nanoparticles may be positively charged nanoparticles. In particular embodiments of the invention, the gold nanoparticles may have one or more of the following properties:
(ai) an absorption peak at about 527 nm;
(aii) an average diameter size of about 13 nm as determined from transmission electron microscopy;
(aiii) a zeta potential in an aqueous solution of from +20 to +30 mV, such as about +24.5 mV; and
(aiv) infra-red absorption peaks at about 1650 cm⁻¹ and about 3450 cm⁻¹. Silver nanoparticles with similar properties may also be used.

In embodiments of the invention where the nanoparticles having peroxidase-like activity or the peroxidase are gold nanoparticles, they are present in the first and second analyte assay formulations at a concentration of from 0.02 to 0.8 nM, such as about 0.45 nM. A similar concentration may be used for silver nanoparticles.

In embodiments of the invention where the near infra-red absorbing and photothermally-responsive compound capable of forming a charge transfer complex or nanoparticle aggregates is 3,3′,5,5′-tetramethylbenzidine, it may be present in the first and second analyte assay formulations at a concentration of from 0.5 to 3 mM, such as about 1.5 mM.

The glucose oxidase may be present at any suitable concentration in the first and second analyte assay formulations. For example, the glucose oxidase may be present in an amount of from 5 U/mL to 25 U/mL in the first and second analyte assay formulations.

The substrate for active myrosinase that provides glucose as a reaction product may be present in any suitable amount in the first and second analyte assay formulations. For example, the substrate for active myrosinase that provides glucose as a reaction product may be present in an amount of from 0.05 mM to 0.75 mM in the first and second analyte assay formulations.

In certain embodiments of the invention, the first and second analyte assay formulations may further comprise an acetate buffer in an amount that provides a pH of from 3.5 to 7.5, such as about 4.5. That is, the pH of the resulting solution should be within the listed pH values.

In certain embodiments of the invention, the sample comprising active myrosinase and the sample comprising inactivated myrosinase may be provided at a concentration of from 1 to 20 mg/mL, such as about 10 mg/mL.

In certain embodiments of the invention, the sample comprising active myrosinase may be subjected to incubation for a period of time (e.g. from 10 minutes to 1 hour, such as about 30 minutes) at a suitable temperature (e.g. from 15 to 30° C., such as about 25° C.) before use in the method. This may result in the myrosinase being at a suitable level of activity before the determination of the level of myrosinase takes place.

The method disclosed herein is intended to allow for the determination of the concentration of active myrosinase in a sample. This method relies on a cascade of reactions to provide the final result. This cascade may be summarized as:
(I) myrosinase (from the sample) is intended to hydrolyse the corresponding substrate (e.g., sinigrin) to produce glucose in the analyte sample;
(II) the glucose oxidase turns glucose into hydrogen peroxide;
(III) a peroxidase (e.g., horseradish peroxidase) or nanoparticles that possess peroxidase-like activity (e.g., positively-charged gold nanoparticles or silver nanoparticles) utilise the hydrogen peroxide to generate a colorimetric effect on a NIR-II absorbing and photothermally-responsive compound (e.g. a charge-transfer complex (such as 3,3′,5,5′-tetramethylbenzidine (TMB), tetracyanoquinodimethane (TCNQ), F4TCNQ, perylene, or combinations thereof such as TMB-F4TCNQ and TMB-TCNQ) or nanoparticle aggregates (e.g., phenylboronate decorated gold nanoparticles)).

The resulting colour/thermal shifts can then be read as a way to determine the concentration of myrosinase. As mentioned previously, the samples may contain endogenous glucose. As such, the analyte reference sample is used to provide a background level of glucose in the system, which may then be used to remove the influence of endogenous glucose (i.e. glucose not generated by myrosinase) from the analyte sample, thereby allowing for the determination of the concentration of (active) myrosinase in the sample.

The determination step of the method may make use of one or more of the following:
(i) Red Green Blue colour values in the analyte sample and Red Green Blue colour values in the analyte reference sample, calculating the Red/Blue ratio for each of the analyte sample and the analyte reference samples and calculating the difference between the Red/Blue ratios to provide a sample Red/Blue ratio and comparing the sample Red/Blue ratio obtained to a pre-determined calibration curve of active myrosinase concentration based on Red/Blue ratios obtained from known concentrations of active myrosinase;
(ii) an infra-red image of the analyte sample and an infra-red image of the analyte reference sample obtained following irradiation of the analyte sample and the analyte reference same by a laser for a third period of time and calculating the temperature difference between a temperature obtained from the infra-red image of the analyte sample and a temperature obtained from the infra-red image of the analyte reference sample and comparing the difference value obtained to a pre-determined calibration curve of active myrosinase concentration based on temperatures obtained from infra-red images of known concentrations of active myrosinase;
(iii) an absorbance spectra of the analyte sample and an absorbance spectra of the analyte reference sample and calculating the difference between the absorbance spectra of the analyte sample and the absorbance spectra of the analyte reference sample and comparing the difference value obtained to a pre-determined calibration curve of active myrosinase concentration based on absorbance spectra of known concentrations of active myrosinase; and
(iv) a temperature signal obtained following irradiation of the analyte sample and the analyte reference sample by a laser for a third period of time and calculating the difference between the temperature signal of the analyte sample and the temperature signal of the analyte reference sample and comparing the difference value obtained to a pre-determined calibration curve of active myrosinase concentration based on temperature signals of known concentrations of active myrosinase.

For (i), a series of known concentrations of myrosinase may be subjected to the same Red Green Blue (RGB) colour value determinations to generate a Red/Blue ratio for each known concentration. These reference samples may then be used to establish the relationship and formula for calculation, so as to essentially provide a suitable calibration curve. Having obtained the calibration curve, the analyte sample and the analyte reference samples are tested and a difference calculation may be performed to provide the actual Blue/Red ratio. The formula used is:

$$c = 1113.36734 \times \frac{B}{R} - 826.72722 \times \frac{{B}^{2}}{R} + 20 \; 4.84169 \times \frac{{B}^{3}}{R} - 49 \; 1.7 \; 0739$$

$${r}^{2} = 0.9497$$

B refers to the Blue value from Red Green Blue (RGB) colour value of obtained image. R refers to the Red value from Red Green Blue (RGB) colour value of obtained image. (B/R)² is the square of Blue/Red ratio. (B/R)³ is the cube of Blue/Red ratio.

It is noted that (i) relies only on colourimetric sensing, so only a smartphone with a camera and access to an online calculator is needed. This avoids the need for more expensive commercial equipment and allows for a direct readout to be obtained with intelligent assisted data processing (as explained in more detail in the examples section below).

For (ii), a similar methodology is used, but based on the temperature of the samples following excitation by a laser.

For (iii) and (iv), the difference values may be more directly obtained from the absorbance spectra and temperature signals, respectively.

The results of the determination processes (i) to (iv) (e.g. one or both of (i) and (ii)) may be compared with the true value based on the least squares method and verified with good repeatability.

As will be appreciated, two or more of the determination processes (i) to (iv) may be used to provide a more accurate determination of the concentration of active myrosinase in the sample. This may be achieved by any suitable means.

In embodiments of the invention where one or both of (ii) and (iv) of step (c) are used to determine the concentration of the active myrosinase in the sample, then the laser may be a near infra-red light laser. In such embodiments, one or more of the following may apply:
the laser provides a light beam having a wavelength from 1000 to 1500 nm, such as about 1064 nm;
the laser has a power density of from 0.5 to 3 W/cm², such as about 1 W/cm²; and
   the third period of time is from 20 seconds to 5 minutes.

In embodiments of the invention that may be mentioned herein, the first and second periods of time are from 10 minutes to 1 hour, such as 30 minutes. As will be appreciated, any suitable time may be selected, through calibration may need to be re-run if a different time is selected, as the active myrosinase will produce more or less glucose depending on whether the new timing is longer or shorter than the previous time.

In embodiments of the invention, the absorbance spectra of the analyte sample and the analyte reference sample may be based on their absorbance at a specified wavelength or a wavelength range in the near infra-red range. For example, the specified wavelength may be a wavelength selected from 1,000 to 1,500 nm, such as 1064 nm.

In particular embodiments of the invention that may be mentioned herein, the first and second analyte formulations may comprise:
a sinigrin;
a glucose oxidase;
3,3′,5,5′-tetramethylbenzidine; and
gold nanoparticles.

As will be appreciated, the method disclosed herein can be used to inspect and rate a product, such as dietary supplements and nutraceuticals that claim to have certain levels of myrosinase (e.g. certain such products may claim to have 13 mg of myrosinase enzyme per serving).

In addition, the method may be applied to:
measure myrosinase in dietary supplements, nutraceuticals, semi-processed food and health products;
assess dietary micronutrients of various vegetables for grading and diet nutritional intake; and
evaluate freshness within a specific category (e.g. of batches of particular vegetables).

Thus, in certain embodiments of the invention, the method may further comprise a step (d), which is selected from one or more of the following:
(di) retaining or disposing of a batch from which the sample was drawn based on a level of freshness of the batch based on the concentration of the active myrosinase in the sample; and
(dii) labelling a batch from which the sample was drawn with a specific level or amount of active myrosinase.

In a second aspect of the invention, there is provided a formulation for use in a method of quantitatively determining the concentration of active myrosinase in a sample, the formulation comprising:
a substrate for active myrosinase that provides glucose as a reaction product;
a glucose oxidase;
either a near infra-red absorbing and photothermally-responsive compound capable of forming a charge transfer complex or nanoparticle aggregates; and
   either a peroxidase or nanoparticles having peroxidase-like activity.

As this formulation (which corresponds to the first and second analyte assay formulations of the first aspect of the invention) has been described above, the various possible embodiments as described hereinbefore will not be repeated here for the sake of brevity.

Particular formulations according to this aspect may comprise:
sinigrin;
a glucose oxidase;
3,3′,5,5′-tetramethylbenzidine; and
gold nanoparticles.

In yet a further aspect of the invention, there is provided a kit of parts suitable to provide a formulation as described herein, wherein the kit of parts comprises:
a substrate for active myrosinase that provides glucose as a reaction product, optionally wherein the substrate for active myrosinase that provides glucose as a reaction product is sinigrin;
a glucose oxidase;
either a near infra-red absorbing and photothermally-responsive compound capable of forming a charge transfer complex or nanoparticle aggregates; and
either a peroxidase or nanoparticles having peroxidase-like activity. As said formulation and the components are described in detail hereinbefore, a discussion is omitted here for the sake of brevity.

Advantages associated with the disclosed technology are summarized in the numbered clauses below.
1. The sensing technology disclosed herein allows for a simpler, more cost-effective and less labour-intensive onsite dietary myrosinase profiling test procedure. The sample screening can be easily achieved with lower technical input with intelligent processing and visual output.
2. As an example of a chromogenic substrate, TMB was selected in the examples below because of its excellent sensitivity, favorable stability, and anti-interference. As noted, it also has a dual function due to its ability for simultaneous colorimetric and photothermal conversion, which enables dual channel screening.
3. As noted herein, a nanomaterial-based mimic enzyme can be used to mediate colorimetric assays with higher stability, lower cost, wider working temperature and pH range than corresponding natural enzymes (e.g., horseradish peroxidase, HRP). For example, the working pH range may be from 3.5 to 9.0 for the nanomaterial-based mimic enzyme compared to a working pH range of 5.0 to 8.0 for a natural enzyme. In embodiments where this mimetic enzyme is used, it may contribute to a more conducive onsite profiling.
4. The absorbance of chromogenic agent (e.g. TMB) at 1064 nm (i.e. in NIR-II) may significantly exclude the interference signal from impurity of plant samples, which greatly expands the application range of screening system.
5. Protein extraction and purification is avoided which is beneficial to reduce cost and greatly shorten the sample preparation time.
6. With the assistance of smartphone readout, the colorimetric images could be intelligently processed by smartphone to obtain their RGB values, which can realize rapid onsite myrosinase detection with good results according to the relationship functions between B/R values and myrosinase concentration.
7. Temperature signals and infrared thermal images generated from photothermal effect of a charge transfer complex may improve detection credibility further, which make the results more reliable.
8. Visual probe enables smartphone scanning for intelligent processing of RGB ratio allowing fast and reliable output.
9. This sensing technology has been successfully utilized on a wide range of real samples including food raw material, dietary supplements, condiments, and bacteria.

Further aspects and embodiments of the invention will now be discussed by reference to the following non-limiting examples.

### EXAMPLES

### Materials

Gold(III) chloride trihydrate (HAuCl₄·3H₂O), chitosan (low viscosity, <200 mPa s), Sinigrin (Sin), myrosinase (Myr), horseradish peroxidase (HRP), glucose oxidase (GOx), and a commercial GO detection kit were obtained from Sigma-Aldrich. TMB, H₂O₂ (3%), acetic acid, sodium acetate, KH₂PO₄, KCl, Na₂HPO₄, and NaCl were purchased from Tokyo Chemical Industry Co., Ltd. (Tokyo, Japan). BroccoMax was purchased from Jarrow Formulas (Singapore). Dualspices Japanese wasabi powder was purchased from iHerb (America). Cruciferous vegetables (broccoli, cauliflower, cabbage, Chinese cabbage, Xiao Bai Cai, etc.) and non-cruciferous vegetables (lettuce and carrot) were purchased from the local supermarket. All chemical reagents were of analytical reagent grade and used as received without further treatment. Milli-Q water was used to prepare all solutions.

### Analytical Techniques

### Ultraviolet-Visible-Near Infrared (UV-Vis-NIR) Spectroscopy

UV-vis-NIR absorption spectra were gauged by a Perkin Elmer Lambda 950 spectrometer.

### Transmission Electron Microscopy (TEM)

The micromorphology of the prepared AuNPs was characterized by TEM on an FEI EM208S TEM (Philips) with an accelerating voltage of 100 kV.

### Dynamic Light Scattering (DLS) Measurement and Zeta Potential

Dynamic light scattering (DLS) measurement and zeta potential were estimated on a Brookhaven 90 Plus nanoparticle size analyzer.

### Fourier Transform Infrared (FTIR) Spectroscopy

FTIR spectroscopy was performed on a Bruker Vertex 80v vacuum FTIR spectrometer.

### Temperature Changes and Infrared (IR) Thermal Images

The temperature changes and infrared thermal images were obtained by an IR thermal camera (Forward Looking InfraRed handheld thermal camera (FLIR)).

### Example 1. Rapid and Simple NIRII Photothermal Responsive Onsite Myr Detection Via Smartphone Readout

We explored a novel sensor that combines the photothermal effects and colorimetric signals for rapid and precise onsite Myr analysis at the NIR-II window via smartphone readout (FIG. 1). Briefly, Sin, Myr and GOx were first mixed for 30 min to generate H₂O₂, then TMB and AuNPs were added, incubated at 45° C. for 25 min. After cooling to room temperature, the solution changed from colorless to blue due to the formation of charge transfer complex (CTC), which possessed broad absorption from UV to NIR-II window. Furthermore, the temperature signals and infrared thermal images would be obtained caused by CTC's photothermal effect after irradiated by 1064 nm laser. Thus, the combined detection of Myr by colorimetric and temperature was achieved. Further, for the purpose of detecting Myr in actual samples intelligently, we exploited a smartphone-based website named “Calculator” to obtain the relevance between the B/R values of colorimetric images and Myr concentrations. Then, the Myr concentrations in actual samples would be determined according to the formula via smartphone readout. Specific details are provided in the following examples.

### Example 2. Preparation of AuNPs

AuNPs were prepared according to previous procedures with minor modifications (Wu, L. et al., J. Phys. Chem. C 2007, 112, 319-323).

Briefly, 0.228 mL of 25.39 mM HAuCl₄ and 4 mL of 2.254×10⁻³ g/mL chitosan in diluted acetic acid (V_{CH3COOH}:V_{H2O}=1:9) were added to a flask with 21.772 mL of H₂O. Then, the mixture was heated to 120° C. with vigorous stirring and the color of the solution turned to red after 5 min, and the mixture was refluxed for another 25 min at 120° C. under vigorous stirring. The AuNPs sample can be used after the solution cooled to room temperature. For evaluating the effects of the different preparation times on AuNPs' catalytic activity, the mixture was allowed to continue to react for 0, 5, 35 and 55 min after the solution turned red.

### Example 3. Effect of Preparation Time of AuNPs on Catalytic Activity

The effect of preparation time on the peroxidase-like (POD-like) activity was examined to obtain AuNPs with the greatest catalytic activity. It can be seen from FIG. 2 that after 10 min of reaction, a low-intensity peak at 1064 nm appeared, and the intensity increased with increasing reaction time, and that the intensity decreased after 30 min; the absorbance at 1064 nm represents the extent of oxidization from TMB to CTC and the POD-like ability of AuNPs. Thus, the activity drops when the time exceeds 30 min due to the aggregation of AuNPs at high temperature over time. Therefore, 30 min was selected as the optimal preparation time for AuNPs.

### Example 4. Characterisation of AuNPs

Characterization of AuNPs was performed after obtaining AuNPs with the highest catalytic activity.

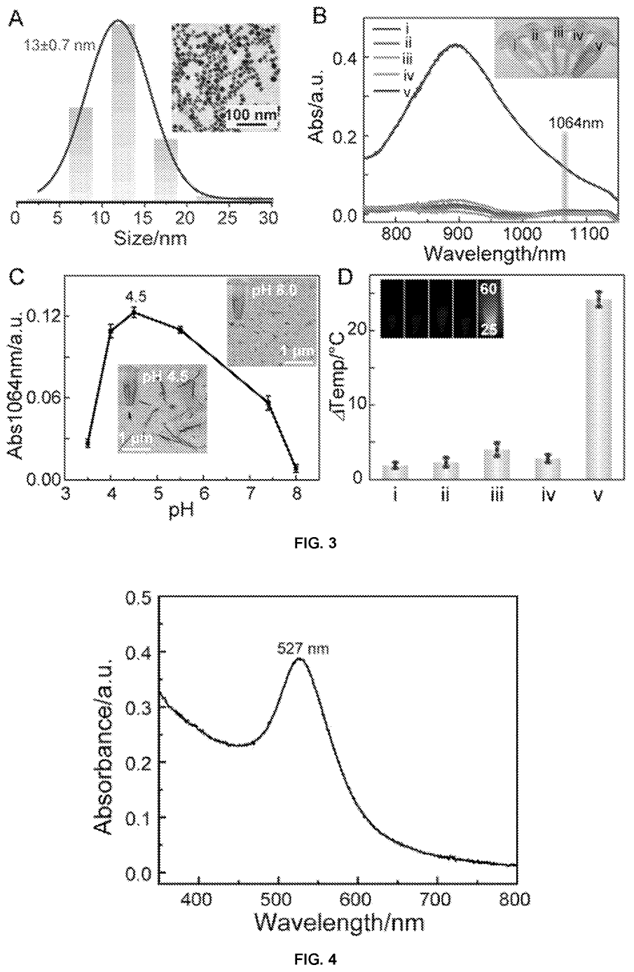

The morphology and size distribution of the prepared AuNPs were first characterized by TEM. According to the inset in FIG. 3A, the average diameter is approximate ˜13 nm, the zeta potential of the solution was measured to be +19.2 mV, and the positive charge on the particles facilitates their dispersion throughout the medium. FIG. 4 suggests that AuNPs possessed a strong absorption peak at 527 nm, which is nearly consistent with previous studies (Wang, Z. et al., Nano Res. 2019, 12, 49-55). According to the formula c=A₄₅₀/ε₄₅₀ (ε₄₅₀=2.18×10⁸) reported by Ray et al. (Darbha, G. K. et al., J. Am. Chem. Soc. 2008, 130, 8038-8043), the AuNPs concentration was estimated to be 4.5 nM.

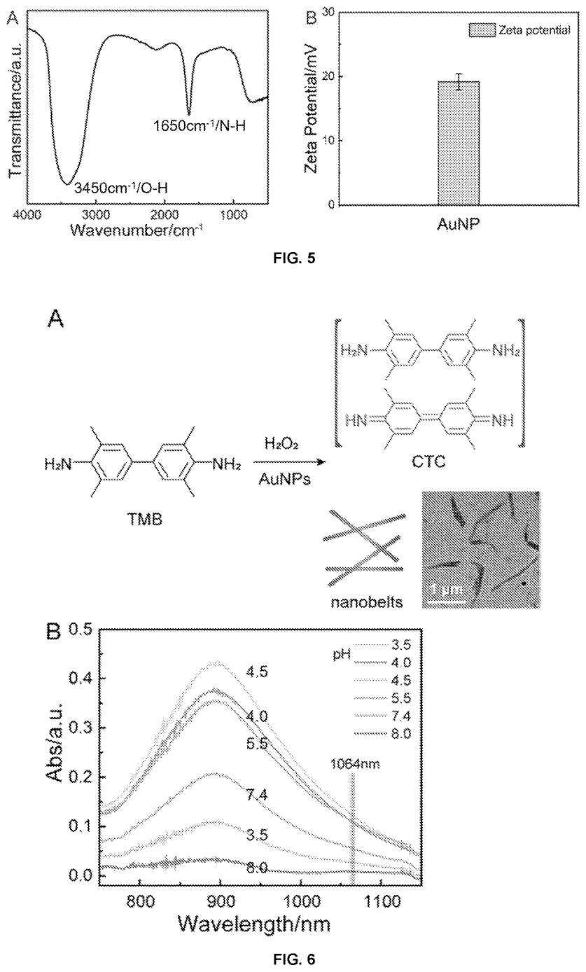

Furthermore, FTIR was employed to obtain absorption bands resulting from the surface functional groups. FIG. 5 depicts that AuNPs possessed a positive charge; the bands at 1650 and 3450 cm⁻¹ were assigned to the existence of chitosan, and they represent the amino group and hydroxyl group, respectively, which were pivotal for the stability in the aqueous solution (Wu, L. et al., J. Phys. Chem. C 2007, 112, 319-323). These results demonstrate that the synthesized AuNPs have potential to act as a POD-like enzyme.

### Example 5. Verification of NIR-II Photothermal and Colorimetric Sensing Based on the AuNP-Catalyzed TMB and H₂O₂ System

The detection of commercial H₂O₂, GO, and Myr was used to confirm the viability of the proposed method.

### Results and Discussion

First, a variety of known concentrations of Myr were mixed with sinigrin (Sin) and GOx to produce a series of solutions containing varying amounts of H₂O₂. Upon addition of TMB and AuNPs to the solution, H₂O₂ would be decomposed to ·OH, thanks to the catalysis of AuNPs, and TMB would be oxidized and transformed into CTC with a blue color and broad absorption from the ultraviolet to NIR-II region (Jiang, C. et al., RSC Adv. 2017, 7, 44463-44469).

In order to illustrate the NIR-II-driven photothermal and colorimetric effect of the AuNPs-TMB-H₂O₂ system, the absorption spectrum of the system with different components was collected, as shown in FIG. 3B, and a broad absorption spectrum extending to the NIR-II region emerged (line (v)) accompanied by a significant color change from colorless to blue (v in the inset) only in the presence of AuNPs, H₂O₂, and TMB. As shown in FIG. 3B and its inset, the method can successfully detect H₂O₂. Also, as control, no obvious absorbance and color changes were observed in other situations without the involvement of AuNPs, TMB, or (i)-(iv). Typically, TMB oxidized by H₂O₂ contributes to the assembly of long straight nanobelt structure CTC with a broad absorption spectrum from the UV to NIR-II region (Wang, Z. et al., Nano Res. 2019, 12, 49-55; and Wang, Z. et al., ACS Nano 2019, 13, 5816-5825), as exhibited in FIG. 6A and the bottom left inset of FIG. 3C.

Furthermore, CTC formation was sensitive to changes of pH value with the color changing from blue to green. As shown in FIGS. 3C and 6B, the absorbance at 1064 nm increased first and then declined with an increase in pH with the most satisfactory result at pH 4.5, and the morphology changed to short nanofragments (top right inset) under higher pH values (e.g. pH=8.0) due to the generation of yellow diamine as confirmed by our previous studies (Wang, Z. et al., Nano Res. 2019, 12, 49-55; and Wang, Z. et al., ACS Nano 2019, 13, 5816-5825).

Then, the temperature signals and infrared thermal images were acquired as a result of CTC absorbance at 1064 nm, which could enhance the detection accuracy. The samples were irradiated by a 1064 nm laser with a power of 1 W/cm² for 5 min, and the FLIR was used to record the temperature changes (FIG. 3D); the temperature elevated by 24.2° C. preeminently only when the system contains H₂O₂, AuNPs, and TMB, and corresponding infrared thermal images are displayed in inset (v). On the other hand, there was no dramatic temperature rise in the other systems (i-iv) with missing components. The results revealed that the response occurred only in the presence of both H₂O₂ and AuNPs, indicating that the synthesized AuNPs possess POD-like activity, and the system possessed the ability of NIR-II absorption-driven photothermal and colorimetric sensing for H₂O₂ due to the formation of CTC.

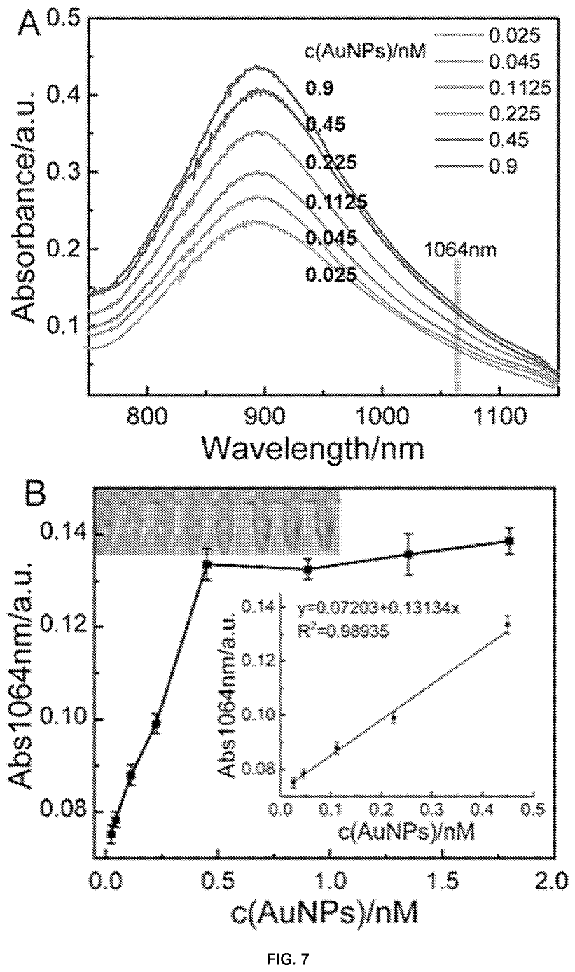

The POD-like activity of AuNPs was also investigated by changing the AuNPs concentration. FIG. 7A demonstrates that the absorbance at 1064 nm increased as the mole concentration of AuNPs increased from 0.025 to 0.9 nM, and the inset of FIG. 7B displays that the color of the solution deepened as the mole concentration of AuNPs increased; we chose 0.45 nM as the optimal concentration of AuNPs in the following steps. The slope of the plot line is 0.13134 (inset of FIG. 7B).

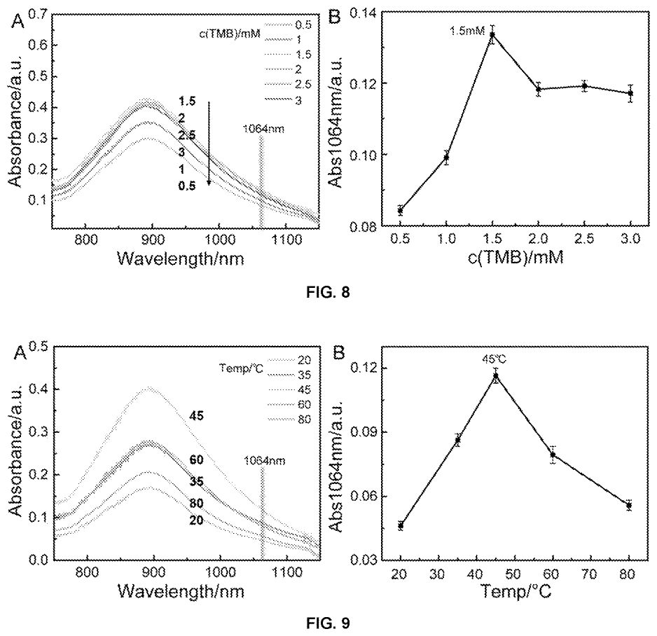

In this system, TMB was used to produce the photothermal agent CTC, so it is vital to optimize the TMB concentration. As shown in FIG. 8A, the absorbances at 1064 nm increased in the concentration range of 0.5 to 1.5 mM and decreased at higher concentrations. FIG. 8B indicates that the yields of CTC improved with increasing substrate concentrations, reaching a maximum at 1.5 mM TMB concentration. While TMB levels were in excess, a slight color fading was observed, which may be attributed to the formation of oxTMB²⁺ with a light-yellow hue as a result of further oxidation (Gao, L. et al., Nat. Nanotechnol. 2007, 2, 577-583). Therefore, 1.5 mM was the optimal TMB concentration in our system.

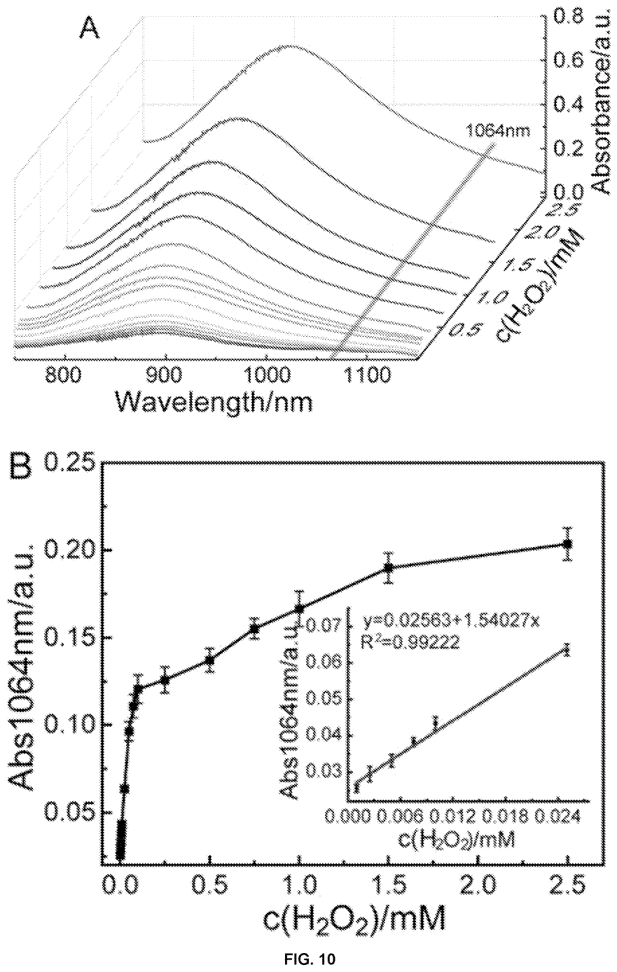

Then, we also investigated the impact of temperature on the activity of AuNPs, where FIGS. 9A-B demonstrate that the absorbance at 1064 nm increased with the increase of temperature within the range of 20 to 45° C. and then decreased from 45 to 80° C.; thus, 45° C. was chosen for the following experiments. It is worth noting that the AuNPs retain peroxidase activity even when heated to 80° C. This is superior to the natural HRP enzyme that loses activity once the temperature exceeds 50° C. (Pardini, A. et al., Food Chem. 2021, 355, 129634). This may be due to the external polymer chitosan being more resistant to high temperatures than small molecules. After optimizing all conditions, their ability to detect commercial H₂O₂ was first evaluated, and the results in FIG. 10 illustrate that the system could detect H₂O₂ from 1 μM to 2.5 mM with a LOD of 0.68 UM, comparable to the results published previously (Jiang, T. et al., Chem. Commun. 2009, 15, 1972-1974).

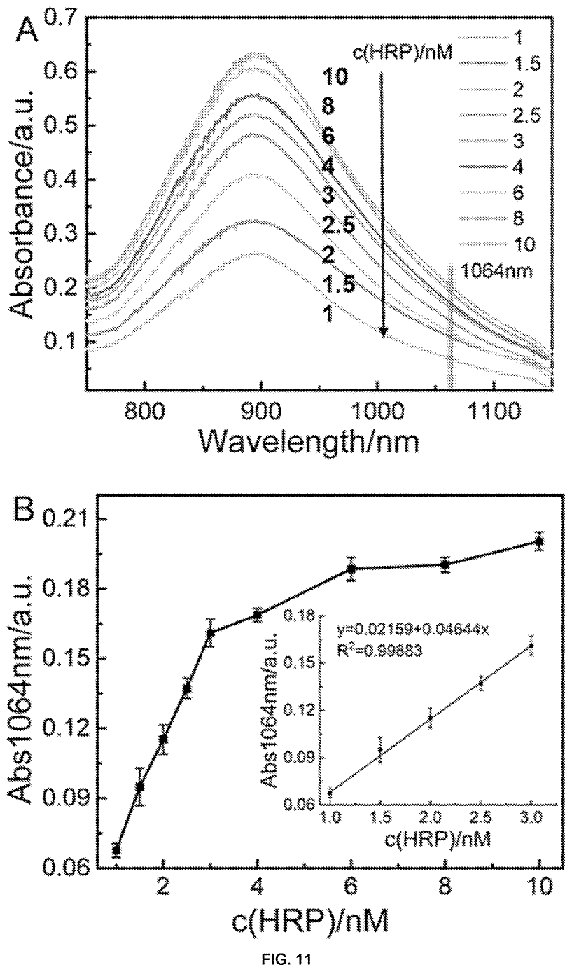

In contrast, the LOD and slope of HRP were 0.47 nM and 0.04644 nM, respectively (FIG. 11). The slope indicates the increment of absorption vs per nanomolar of natural or mimic enzyme. The greater the slope value, the greater POD-like activity.

### Example 6. Optimization of Conditions

### Optimization of Conditions

Firstly, the concentration of TMB was optimized as follows. Different concentration of TMB (0.5, 1, 1.5, 2, 2.5, 3 mM) was mixed with AuNPs and H₂O₂. Then, UV-Vis-NIR spectrometer was employed to obtain the absorption spectrum of AuNPs catalyzed TMB-H₂O₂ system. Secondly, the formation of CTC and the activity of AuNPs were also influenced by pH of the acetate buffer. So TMB, H₂O₂, and AuNPs were added into a range of buffers with various pH (3.5, 4, 4.5, 5.5, 7.4, 8.0), then collected the absorption spectra of different samples after cooling to room temperature. Finally, for investigating the effects of incubation temperature, TMB, H₂O₂, and AuNPs were added into acetate buffer, then the mixture was respectively incubated at 20° C., 35° C., 45° C., 60° C., 80° C., to obtain the absorption spectra.

### Results and Discussion

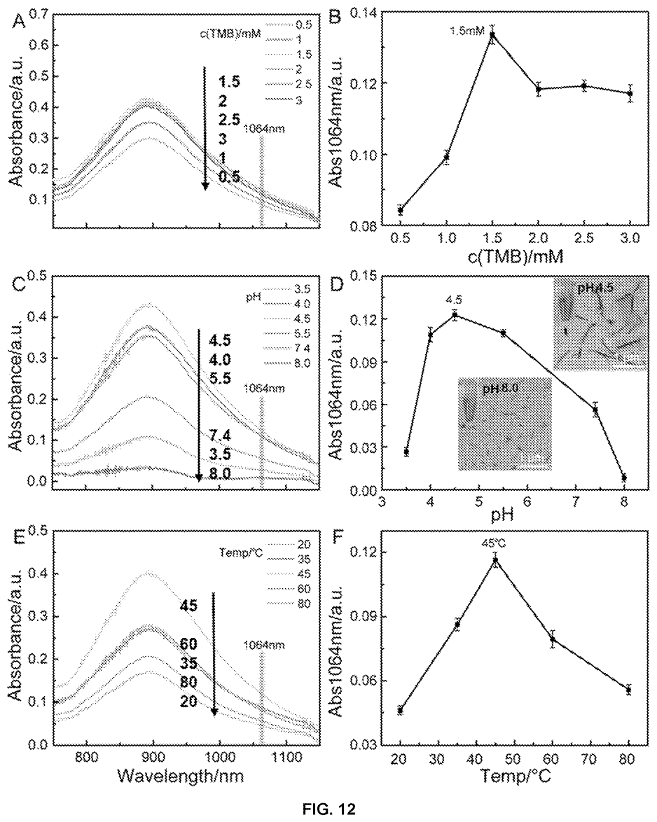

In the nanoenzyme-mediated photothermal biosensing platform, TMB was used to produce the photothermal agent CTC, so it is vital to optimize the TMB concentration for optimal detection performance. Generally, given a constant AuNPs concentration (0.45 nM) and reaction time (25 min), a series of TMB concentrations ranging from 0.5 to 3 mM were evaluated. As shown in FIGS. 12A-B, the absorbances at 1064 nm (representing the characteristic peak of CTC) increased in the concentration range of 0.5 to 1.5 mM and decreased at higher concentrations. The results indicates that, given a fixed amount of catalysts, the yields of CTC improved with increasing substrate concentrations, reaching a maximum at 1.5 mM TMB concentration. While TMB levels were excessive, a slight color fading was observed, which may be attributed to the formation of oxTMB²⁺ with a light-yellow hue as a result of further oxidation (Stefan, L. et al., Nucleic Acids Res. 2012, 40, 8759-8772).

The effects of pH and temperature were evaluated to determine the optimal experiment conditions. To examine the effect of pH, acetate buffers with varied pH ranging from 3.5 to 8.0 were first prepared. Results in FIGS. 12C-D illustrate that the POD-like activity of AuNPs is pH-dependent, and the most satisfactory result was obtained at pH 4.5. Furthermore, CTC, the blue color products, decomposed gradually with an increase in pH, and the morphology changed from long straight nanobelts (top right inset in FIG. 12D) to short nanofragments (bottom left inset in FIG. 12D) under pH 4.5 and pH 8.0, according to previous kinds of literature (Josephy, P. D. et al., J. Biol. Chem. 1982, 257, 3669-3675; and Awano, H. et al., Bull. Chem. Soc. Jpn. 1990, 63, 2101-2103), this is due to the generation of yellow diimine. Concurrently, the solution color changed to green, which is in agreement with our previous results. Then, we also investigated the impact of temperature (20-80° C.) on the activity of AuNPs, FIGS. 12E-F demonstrate that the absorbance at 1064 nm increased with the growth of temperature within the range of 20 to 45° C., and then decreased from 45 to 80° C. AuNPs manifested the supreme POD-like activity at 45° C., so 45° C. was chosen for the following experiments. It is worth noting that the AuNPs retain peroxidase activity even when heated to 80° C. This is superior to the natural HRP enzyme that lose activity once the temperature exceeds 50° C. (Gao, L. et al., Nat. Nanotechnol. 2007, 2, 577-583). This may be due to the external polymer chitosan is more resistant to high temperatures than small molecules.

### Example 7. Photothermal and Colorimetric Bioassay for Myr Measurement

### Photothermal and Colorimetric Assays for Myr Enzyme Detection

A typical spectral photothermal and colorimetric analysis for GO, H₂O₂ and Myr was realized as follows. First, 300 μL of 5 mM TMB in distilled water (ddH₂O), 100 μL of 4.5 nM AuNPs in ddH₂O, and 100 μL of H₂O₂ in ddH₂O with different concentrations were added into 500 μL of acetate buffer (pH=4.5), and then the mixture was incubated in a 45° C. water bath for 25 min and then cooled to the room temperature for adsorption spectroscopy measurement and photothermal analysis.

For GO detection, a mixture of 90 μL GO and GOx (5 mg/mL, 10 μL) was reacted for 30 min. Then, an acetate buffer containing TMB and AuNPs was added and incubated at 45° C. for 25 min. Finally, UV-Vis-NIR spectrometer and Forward Looking InfraRed handheld thermal camera (FLIR) were used to analysis GO.

For Myr detection, Sin (2.5 mM, 30 μL) and GOx (5 mg/mL, 10 μL, according to previous literature (Jiang, C. et al., RSC Adv. 2017, 7, 44463-44469)) were mixed with different concentrations of Myr first, and reacted at 37° C. for 30 min to generate H₂O₂. Then, an acetate buffer containing TMB and AuNPs was added into the aforementioned solution, incubated at 45° C. for 25 min, and cooled to room temperature. Afterward, the solution was used for the analysis of Myr enzyme activity.

### Results and Discussion

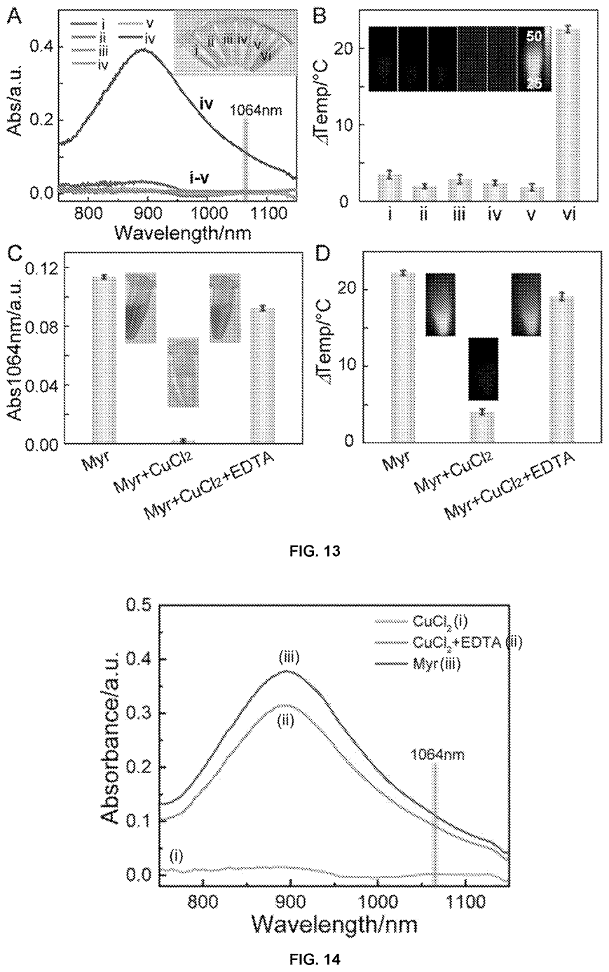

Next, we investigated the feasibility of this method for Myr tests, followed by the optimization of conditions, and the results are shown in FIG. 13A. There are no significant changes both in absorption spectrum and images of samples (i-v, containing incomplete component), whereas an obvious absorbance at 1064 nm emerged in sample vi (including all components) accompanied by a vivid color change from colorless to blue. In agreement with the aforementioned results, H₂O₂ only emerged in the presence of Myr and GOx and then interacted with TMB under the catalysis of AuNPs to yield blue CTC with NIR-II absorption; with excellent photothermal conversion performance for temperature signal acquisition. After all samples were treated with a 1064 nm laser, a drastic temperature improvement of nearly 22.5° C. and remarkable infrared thermal image was acquired in sample vi (FIG. 13B) thanks to the excellent photothermal conversion performance of CTC. In contrast, mild temperature fluctuations and negligible images were observed in other samples (i-v). These results suggested that photothermal and colorimetric signals were only derived from the formation of CTC. Moreover, the viability of analyzing Myr was further studied, and the results of FIGS. 13C and 14 indicate that absorbance at 1064 nm declined dramatically after Cu²⁺ was added into the system containing Sin, Myr, GOx, TMB, and AuNPs. However, the intensity recovered significantly after adding EDTA into the system, which could combine with Cu²⁺ and diminish the inhibitory effect of Cu²⁺. The corresponding temperature signals and infrared thermal images are shown in FIG. 13D. Similarly, the increase of temperature notably reduced from 22.1° C. to 3.9° C. and then rose to 19° C., and the infrared thermal images were also altered clearly. All of these results reconfirmed the feasibility of NIR-II absorption-driven photothermal and colorimetric sensing techniques and indicated their potential to analyze Myr in actual samples by exploiting the NIR-II absorption characteristic of CTC.

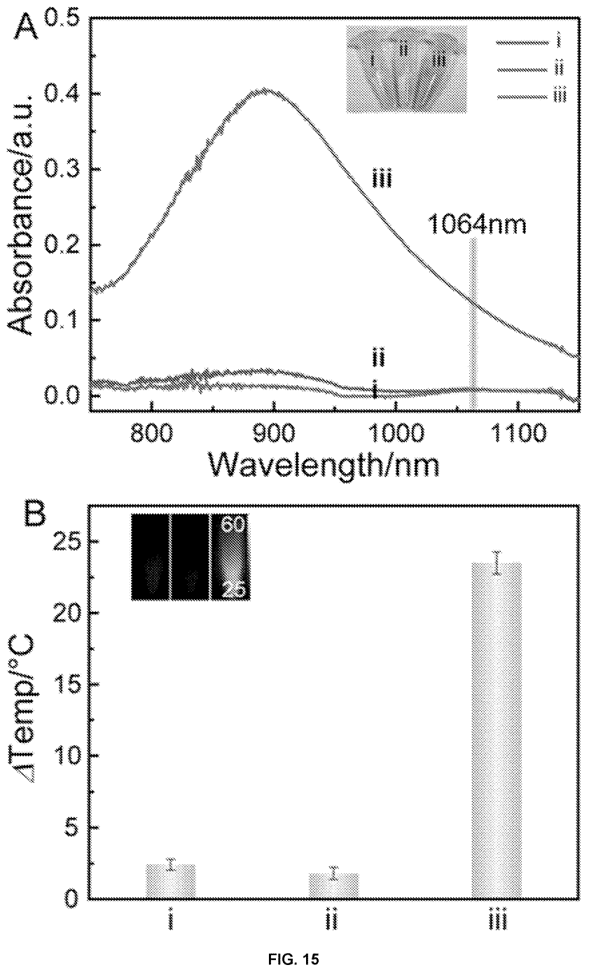

In addition, the successful detection of GO in FIG. 15 also reconfirm the feasibility of the method again.

### Example 8. Quantitative Analysis of the NIR-II Absorption-Driven Photothermal and Colorimetric Sensing for Myr

For the anti-interference experiments discussed below, Sin (2.5 mM, 30 μL) and GOx (5 mg/mL, 10 μL) were mixed with different interfering substances first and reacted at 37° C. for 30 min. Then, acetate buffer containing TMB and AuNPs were added into the above solution, incubated at 45° C. for 25 min, and cooled to room temperature, and afterwards, the solution was used for photothermal and colorimetric analysis.

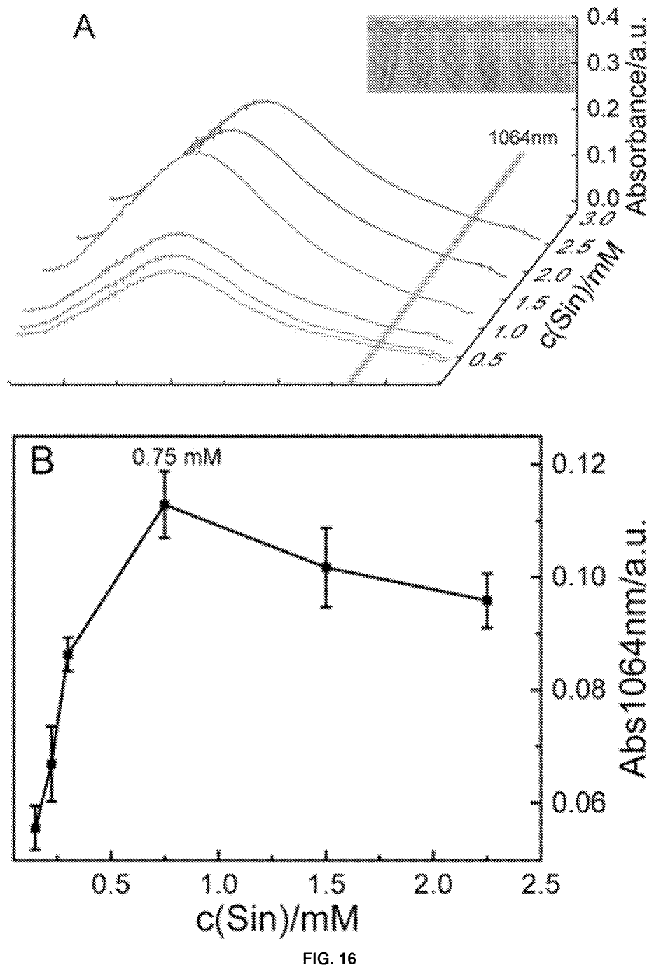

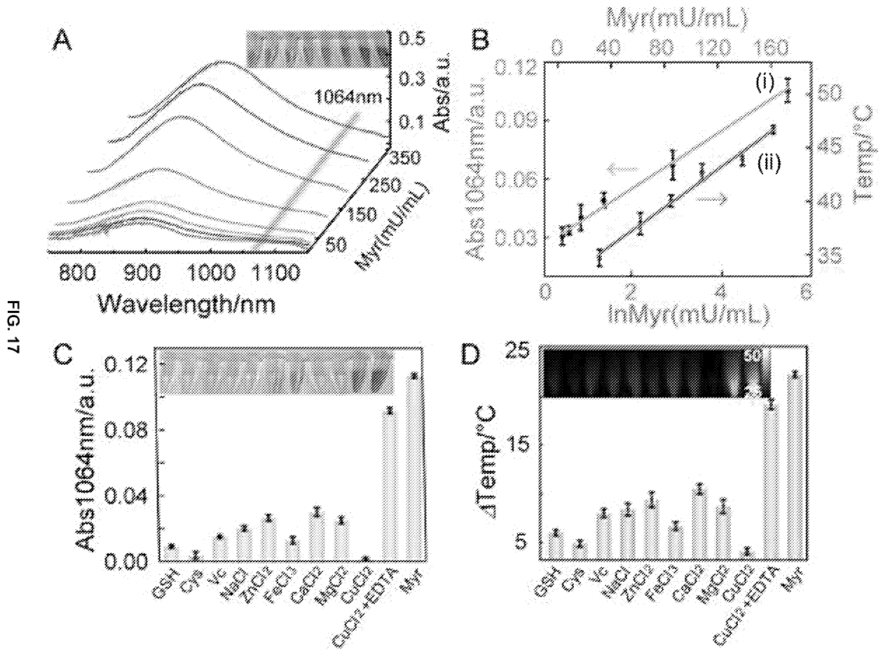

After optimizing all conditions in the above examples, we applied this method to test Myr; however, previous studies indicated that a high GIs concentration has a significant inhibitory effect on Myr activity (Pardini, A. et al., Food Chem. 2021, 355, 129634). Before testing different Myr concentrations, it is necessary to determine the maximum Sin concentration that can be applied. FIG. 16 illustrates that when the concentration of Sin exceeds 0.75 mM, the working efficiency decreases and the blue color becomes faint because excess Sin binds to the active regulatory sites of Myr, thereby inhibiting the activity of Myr to hydrolyze Sin. As a result, 0.75 mM was selected as the maximum Sin concentration for detecting commercial Myr at varying concentrations and constructing a standard curve. As shown in FIGS. 17A-B and 18, when Sin was present at a concentration of 0.75 mM, Myr was detectable in the range of 0-345 mU/mL, and its linear detection range in the absorption spectrum was 0 to 172.5 mU/mL with an LOD of 2.96 mU/mL. Comparatively, our method shows superiority in sensitivity with an LOD lower than that of previously reported work (Finiguerra, M. G. et al., J. Agric. Food Chem. 2001, 49, 840-845; and Gonda, S. et al., Molecules 2018, 23, 2204). Additionally, the linear relationship between temperature and the logarithm of Myr concentration was also exhibited by line (ii) in FIG. 17B with a similar trend as indicated previously (Fu, G. et al., Anal. Chem. 2018, 90, 5930-5937). These results further validate that the method could be employed to Myr analysis in real samples by both temperature and absorbance variations.

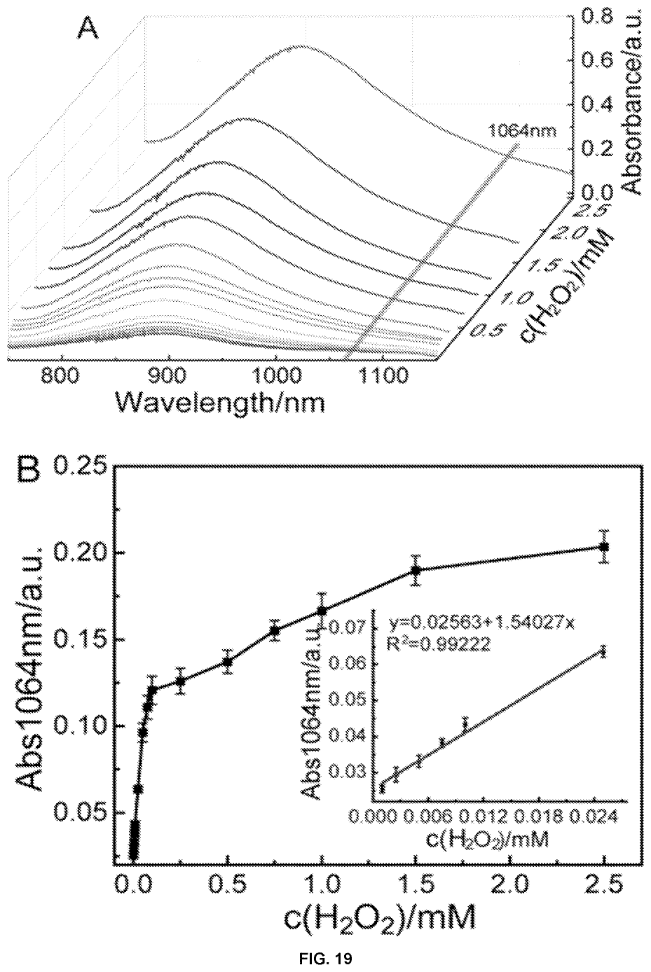

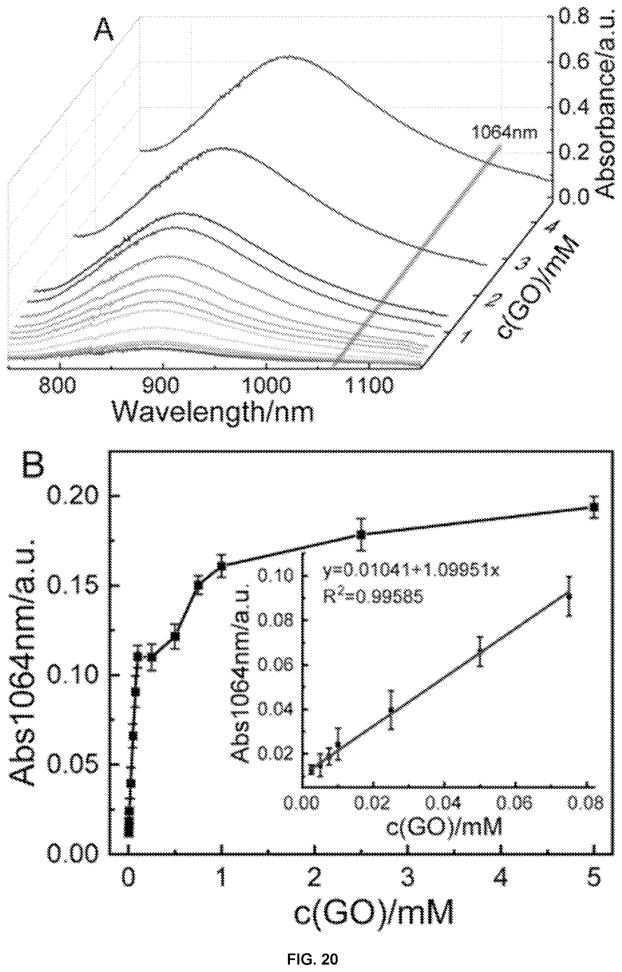

The results in FIG. 19 illustrate that the method could detect H₂O₂ from 1 μM to 2.5 mM, with a linear detection range of 1 μM to 25 μM. GO could be detected in the range of 2.5 μM to 4.5 mM (FIG. 20A) with LOD of 1.67 UM (FIG. 20B). Next, we applied this method to test Myr, however, previous literatures indicate that a high GIs concentration has a significant inhibitory effect on Myr activity (Román, J. et al., Food Chem. 2018, 254, 87-94; and Pardini, A. et al., Food Chem. 2021, 355, 129634).

More importantly, such a simple and rapid method gets rid of complicated sample preparation and measurement processing, therefore providing huge potential for onsite detection. We can also qualitatively distinguish samples of different concentrations of Myr by observing the color with naked eye, which is also beneficial for onsite test.

Moreover, specificity is also an important indicator for evaluating the practicability of an analytical technique, so various interfering factors including amino acids, small molecules, and metal ions were also measured by the NIRII-driven photothermal and colorimetric sensing platform. According to previous reports, high concentrations of Vitamin C (Vc) and NaCl, amino acids, and metal ions can inhibit the activity of Myr (Jwanny, E. W. et al., Phytochemistry 1995, 39, 1301-1303; and Ohtsuru, M. et al., Agric. Biol. Chem. 1979, 43, 2249-2255), while EDTA could alleviate the inhibitory effect of metal ions.

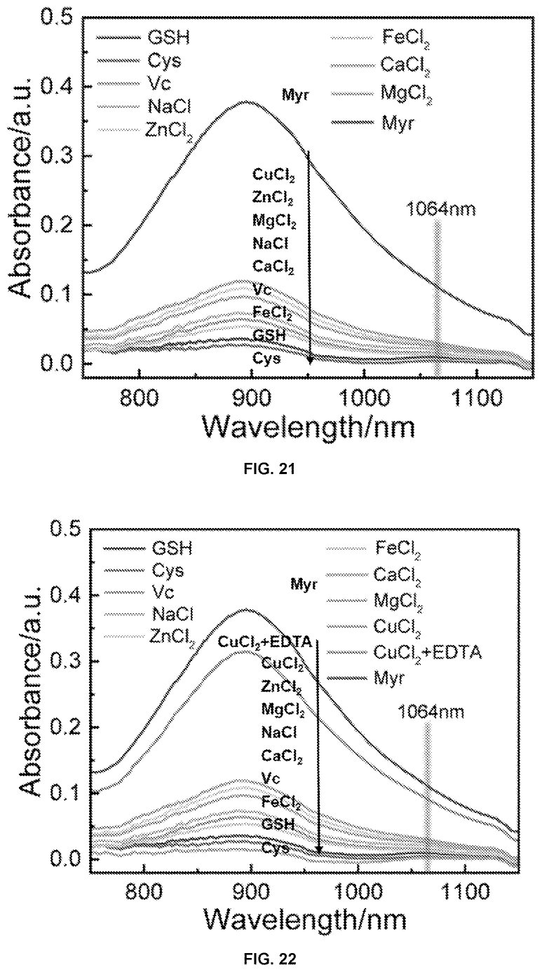

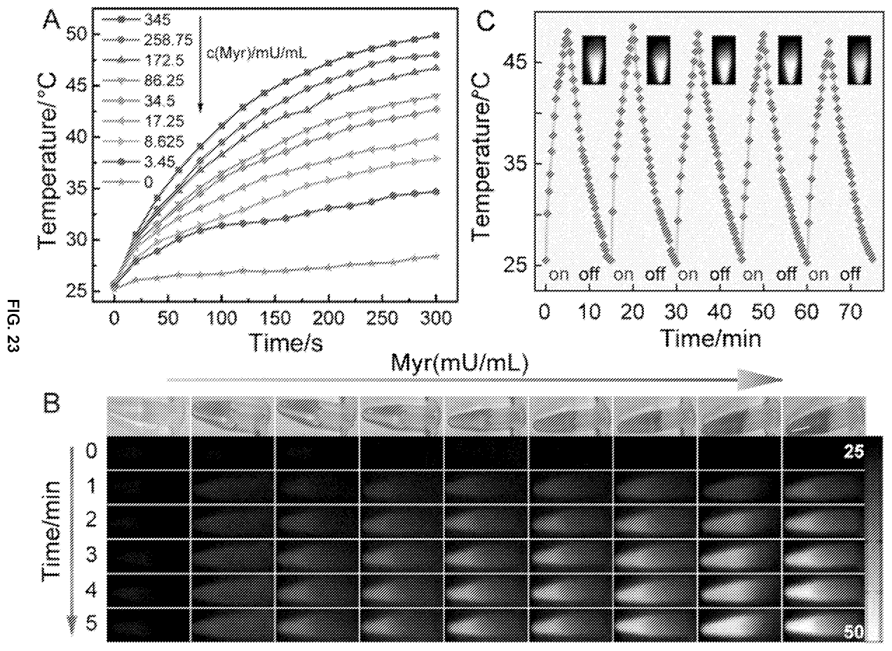

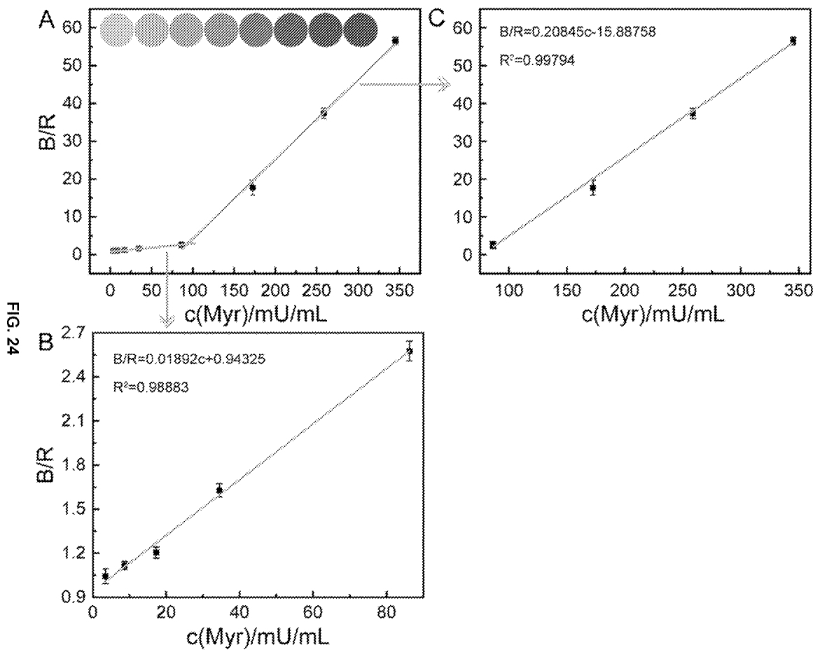

As exhibited in FIG. 17C, we can see that Cu²⁺ has the strongest inhibitory effect on Myr activity, followed by other metal ions, when EDTA and Cu²⁺ coexist in the system, Myr activity is less inhibited due to the combination of EDTA and Cu²⁺, thereby diminishing the inhibitory effect of metal ions. Vc, GSH and Cys also inhibit Myr activity, which is consistent with previous reports. In addition, as exhibited in FIG. 17C, the absorbance at 1064 nm of Myr was notably higher than those of other interfering molecules, and a remarkable color change was observed only for Myr, with the absorption spectrum depicted in FIG. 21. Additionally, FIG. 17D displays the corresponding temperature signals and infrared thermal images, indicating that the temperature elevation of Myr was more apparent than that of other interfering factors after irradiation by a 1064 nm laser. These results imply that the method shows good specificity for the Myr test in a complex matrix environment and potential capacity for the onsite Myr test. Their UV-vis-NIR absorption spectrum is depicted in FIG. 22.

### Example 9. Temperature Response to Myr, Thermal Cycling Stability and Specificity for Myr Test

Next, the temperature response to different concentrations of Myr and thermal cycling stability were discussed, as mentioned above; under the catalysis of AuNPs, TMB reacted with H₂O₂ to produce a blue CTC with a broad absorption peak in the NIR-II region. Taking advantage of the property, the samples were irradiated with a 1064 nm laser to obtain the temperature response of different Myr concentrations, as displayed in FIG. 23A, and we quantitatively analyzed Myr by temperature, for example, the temperature corresponding to 345 mU/mL Myr was approximately 49.8° C. In addition, the corresponding infrared thermal images were captured (FIG. 23B) to improve the precision of this method.

We also evaluated the thermal cycling stability for detecting Myr; as FIG. 23C demonstrates, the method still has a good photothermal response even after 5 cycles of laser irradiation. This denotes that the method proposed in the present disclosure could be utilized for Myr testing with desirable thermal stability and could be used for Myr onsite tests.

### Example 10. Intelligent Onsite Determination of Myr in Actual Samples Via a Smartphone

We planned to examine the feasibility of accurate analysis in our sophisticated and dynamic system. The test results of commercial Myr would be used as a reference to make a preliminary qualitative judgment on the content of Myr in vegetables. Toward this purpose, we applied the method to test Myr in actual samples, and we selected some cruciferous and non-cruciferous vegetables, and the dietary supplements BroccoMax and Dualspices Japanese wasabi powder as the detecting samples.

### Detection of Myr in Actual Samples

Myr in dietary supplements and wasabi was detected by the proposed method as follows: 5 mg/ml of broccoli or cabbage vegetables in ddH₂O or 10 mg/ml of the rest of the actual samples in ddH₂O were heated at 100° C. for 1 h to inactivate Myr; other samples with the same concentration were incubated at room temperature (25° C.) for 30 min, and 90 μL of the samples were mixed with an acetate buffer containing GOx (5 mg/mL, 10 μL), TMB (5 mM, 300 μL), and synthesized AuNPs (100 μL), and incubated for another 30 min. For obtaining temperature signals and infrared thermal images, a 1064 nm laser with 1 W/cm² was used to irradiate these samples after we collected the absorption spectrum.

For Myr analysis in cruciferous plants, we first froze the plant materials using liquid nitrogen for later analysis of Myr (Keck, A. S. et al., J. Agric. Food Chem. 2003, 51, 3320-3327). Then, 5 mg of powdered broccoli or cabbage or 10 mg of powdered vegetables were added into 1 mL of phosphate-buffered saline (PBS) buffer depending on the sample, and heated at 100° C. for 1 h to inactivate endogenous Myr. Then, 60 μL of samples were added into an acetate buffer containing exogenous sinigrin (2.5 mM, 30 L), GOx (5 mg/mL, 10 μL), TMB (5 mM, 300 μL), and AuNPs (100 μL) for 30 min, and we collected their absorbance at 1064 nm, which can be used as a background signal (A_{b}) of various vegetables. Then, the same concentration of samples was incubated at room temperature for 30 min, and acetate buffer containing sinigrin (2.5 mM, 30 μL), GOx (5 mg/mL, 10 μL), TMB (5 mM, 300 μL), and synthesized AuNPs (100 μL) was mixed with 60 μL of samples. The absorbance at 1064 nm was obtained and used as detection signal (A_{d}), and Δ_{A} was used to evaluate the amount of endogenous Myr in the vegetables. In addition, FLIR was used to obtain the temperature signals and infrared thermal images to improve the accuracy of detection.

The background signal (A_{b}) was obtained from the samples treated with 100° C., and the detection signal (A_{d}) was collected from the samples treated at room temperature; the amount of Myr was estimated by subtracting A_{b} from A_{d}, which gives an increased signal (Δ_{A}) as a result. In addition, the FLIR was used to obtain the temperature signals and infrared thermal images. Considering the relationship between spectra, colorimetric images, temperature signals, and infrared thermal images, we correlated the values of the acquired images with the Myr concentrations via the smartphone-based intelligent processing system “Calculator” (see https://calculator-39f49.firebaseapp.com/#/). Typically, by using “Calculator” with a formula, the obtained experimental images will be resolved into digital pixels presented by red, green, and blue colors (RGB) (Stefan, L. et al., Nucleic Acids Res. 2012, 40, 8759-8772). The ratio of different color intensity values, e.g., blue to red (B/R), will be used to validate Myr amounts in real samples.

### Results and Discussion

Considering the relationship between spectrum, colorimetric images, temperature signals, and infrared thermal images, we analyzed the relationship between B/R values of colorimetric images and Myr concentrations via a smartphone-based website called Calculator (https://calculator-39f49.firebaseapp.com/#/), then, using “Calculator” with a formula to achieve intelligent onsite analysis of Myr rapidly. The B/R values were used as a parameter for Myr quantification, and FIG. 24A reveals that the ratio of B and R was increased with the increasement of Myr concentration. From FIG. 24B, the values of B/R displayed a linear relationship with Myr concentration in the range of 0-86.25 mU/mL (R²=0.98883) with LOD 2.48 mU/mL, while the B/R values exceeded 2.57692 (Myr concentration was larger than 86.25 mU/mL), the B/R value was proportional to Myr concentration with R²=0.99794 (FIG. 24C). Therefore, the proposed method possesses promising potential for the determination of Myr within a wide range of 0-345 mU/mL in two consecutive linear ranges. After uploading and decomposing the colorimetric images by “Calculator”, the Myr content in vegetables were gained via smartphone readout.

For the determination of total Myr in vegetables, all samples were flash-frozen in liquid nitrogen, and in order to avoid the interference of the sample itself, we inactivated the sample at 100° C. for 1 h to ensure the inactivation of Myr. Then, the Myr content in the samples treated at room temperature and high temperature was determined using the method proposed in the present disclosure.

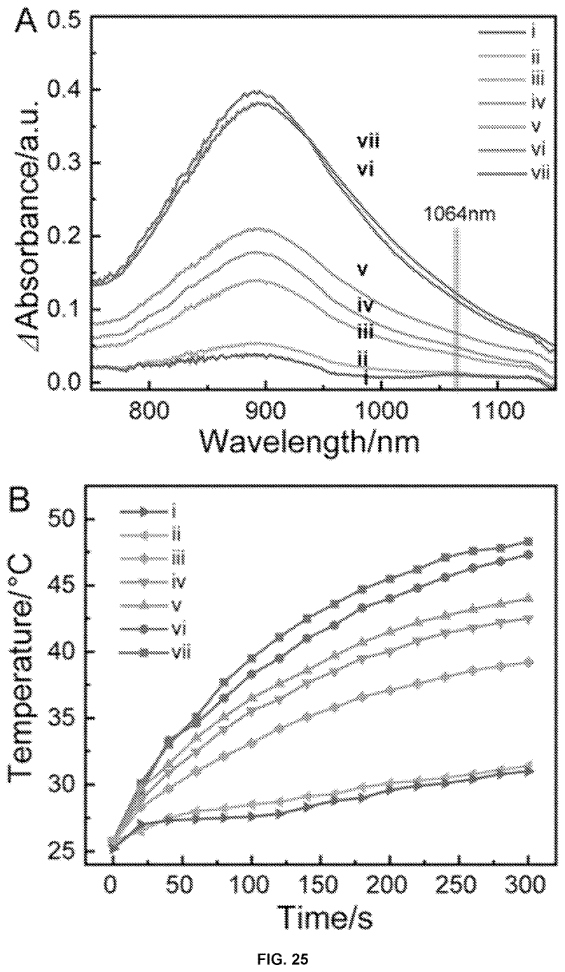

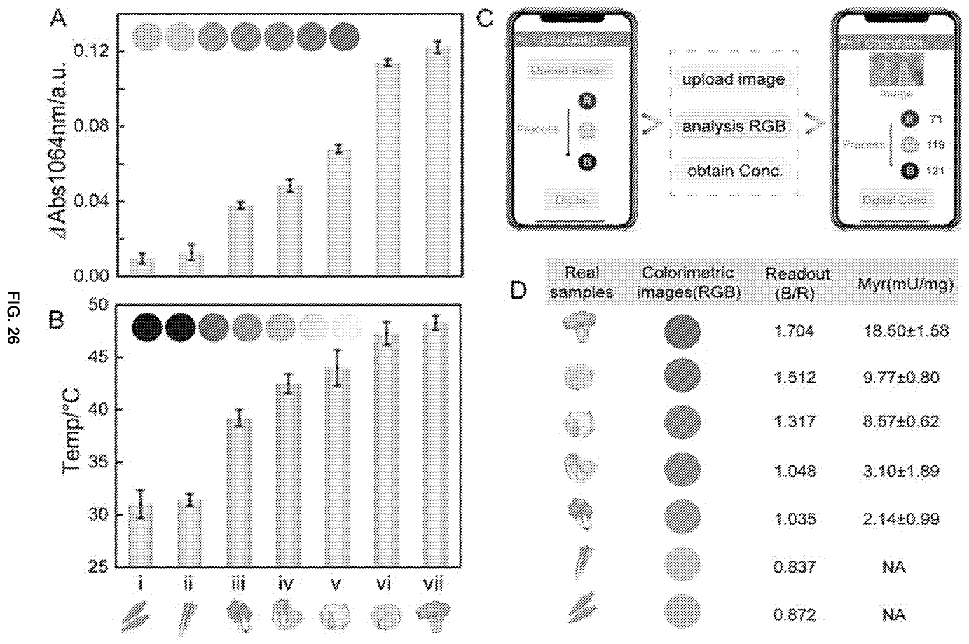

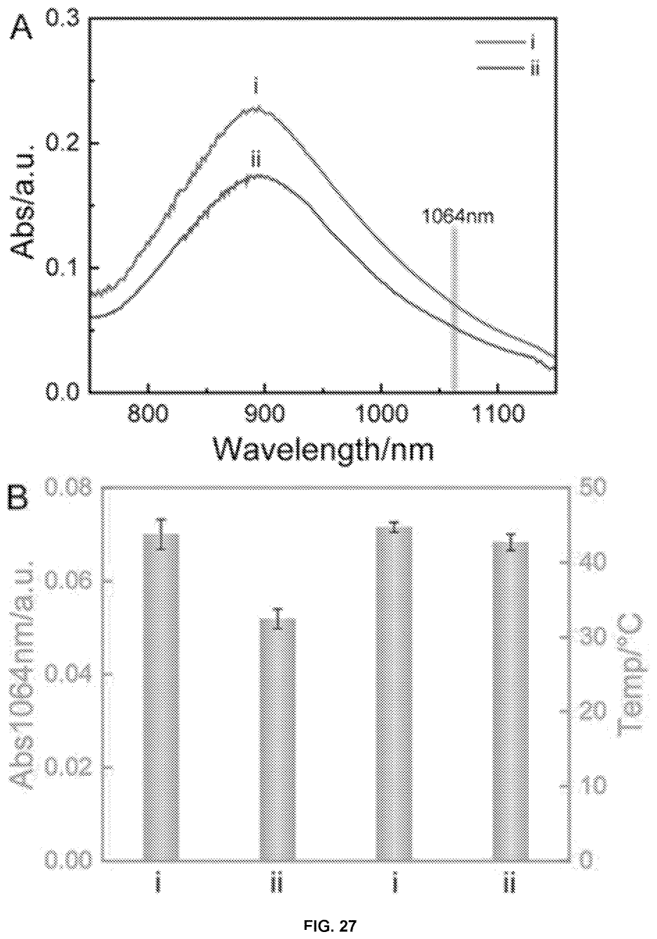

As demonstrated in FIG. 25A, the absorption spectra were obtained by subtracting the absorption spectrum of the samples at high temperature (A_{b}) from the absorption spectrum of the samples at room temperature (A_{d}), named ΔAbsorbance (ΔAbs) spectra. FIG. 26A shows the ΔAbs at 1064 nm of various vegetables including broccoli, cauliflower, green cabbage, Chinese cabbage, Xiao Bai Cai, lettuce, and carrot. Among them, broccoli has the maximum ΔAbs1064 nm value followed by green cabbage, and carrot has the minimum value. Then, their temperature change (FIG. 25B) and infrared thermal images (FIG. 26B) were also acquired by the FLIR. Based on the established calibration curves, the amounts of Myr per mg of actual samples are listed in Tables 1 and 2, obtained from Abs1064 nm and temperature in FIGS. 26 and 27, respectively.

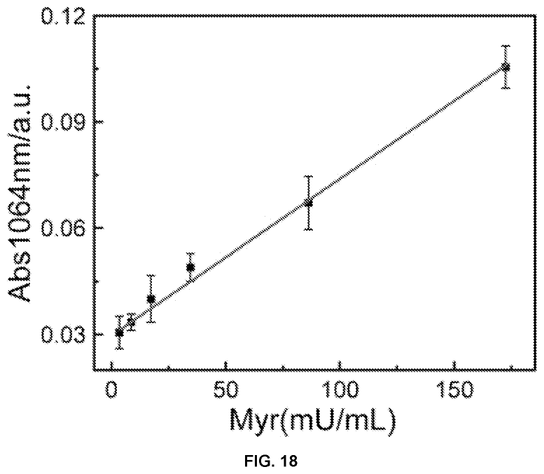

TABLE 1
Myr amounts in per mg actual samples calculated by the calibration
curves of Abs1064nm. The concentration of broccoli and cabbage
was 5 mg/mL, and the concentration of other vegetables was 10
mg/mL. The results were calculated based on the linear relationship
between Abs1064nm and Myr concentration shown in FIG. 18.

| Real | Abs1064nm | Myr |
|---|---|---|
| samples | (a.u.) | (mU/mg) |
| broccoli | 0.07002 ± 0.0032 | 18.32 ± 1.46 |
| cabbage | 0.0519 ± 0.0021 | 10.06 ± 0.96 |
| cauliflower | 0.06711 ± 0.0017 | 8.50 ± 0.39 |
| Chinese Cabbage | 0.04842 ± 0.0033 | 4.24 ± 0.75 |
| XiaoBaiCai | 0.03792 ± 0.0018 | 1.84 ± 0.41 |
| lettuce | 0.01273 ± 0.0041 | NA |
| carrot | 0.00952 ± 0.0026 | NA |

TABLE 2
Myr amounts in per mg actual samples calculated by the calibration
curves of temperature. The concentration of broccoli and cabbage was 5
mg/mL, and the concentration of other vegetables was 10 mg/mL.
The results were calculated based on the linear relationship between
temperature and the logarithm of Myr concentration with fitted equation as
$Temp ( ° C . ) = 2.96 \times lnMyr ( mU / mL ) + 31.42 ( {R}^{2} = 0.98948 ) .$

| | Temp | Myr |
|---|---|---|
| Real samples | (° C.) | (mU/mg) |
| broccoli | 44.7 ± 0.66 | 18.61 ± 1.91 |
| cabbage | 42.7 ± 1.08 | 9.62 ± 0.95 |
| cauliflower | 44 ± 1.7 | 7.51 ± 0.64 |
| Chinese cabbage | 42.5 ± 0.88 | 4.51 ± 0.46 |
| XiaoBaiCai | 39.2 ± 0.79 | 1.46 ± 0.37 |
| lettuce | 31.4 ± 0.56 | NA |
| carrot | 31 ± 1.33 | NA |

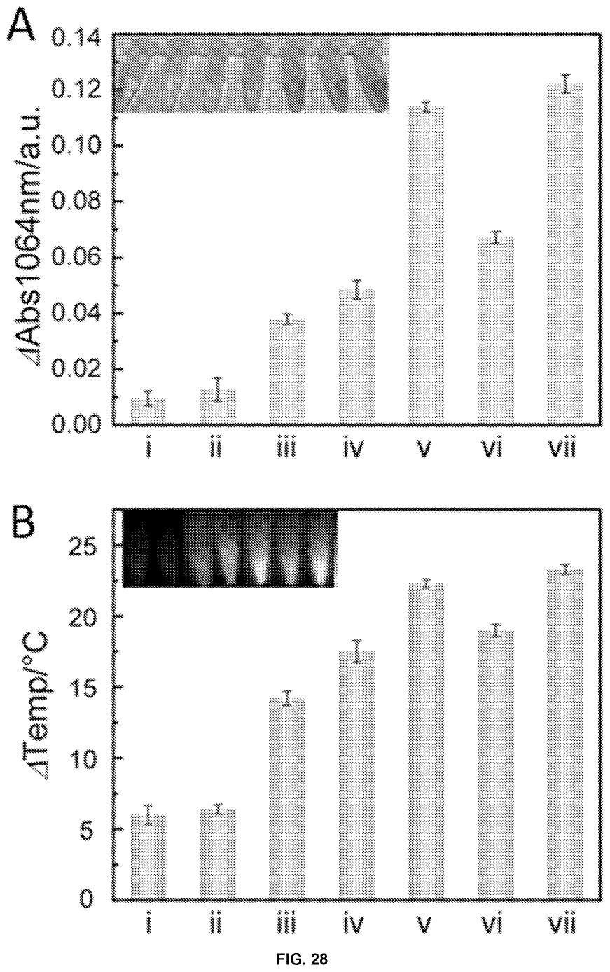

FIG. 28A shows the ΔAbsorbance at 1064 nm of various vegetables including broccoli, cauliflower, green cabbage, Chinese cabbage, Xiao Bai Cai, lettuce, and carrot. Among them, broccoli has the maximum ΔAbs1064 nm value, followed by green cabbage, and carrot has the minimum value. Their infrared thermal images (FIG. 28B) were also acquired by FLIR. The test results of commercial Myr would serve as a reference to make a preliminary qualitative evaluation of the Myr concentration in vegetables.

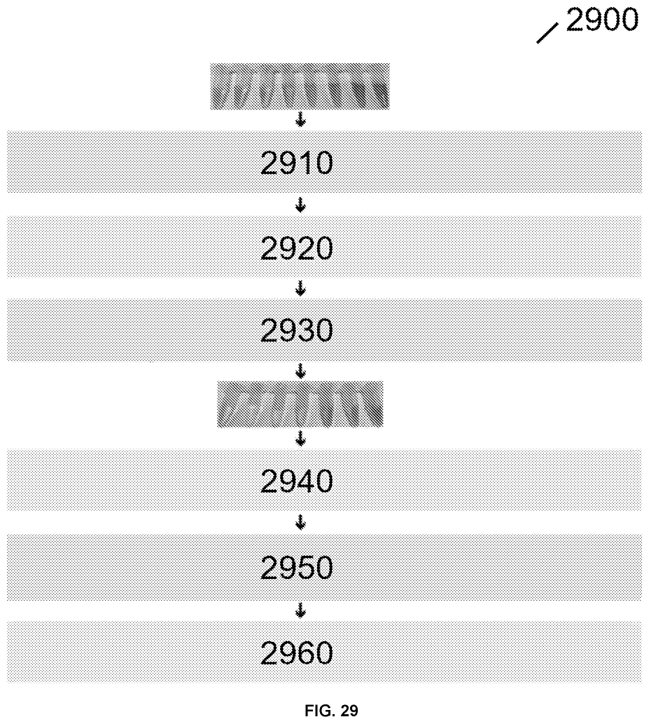

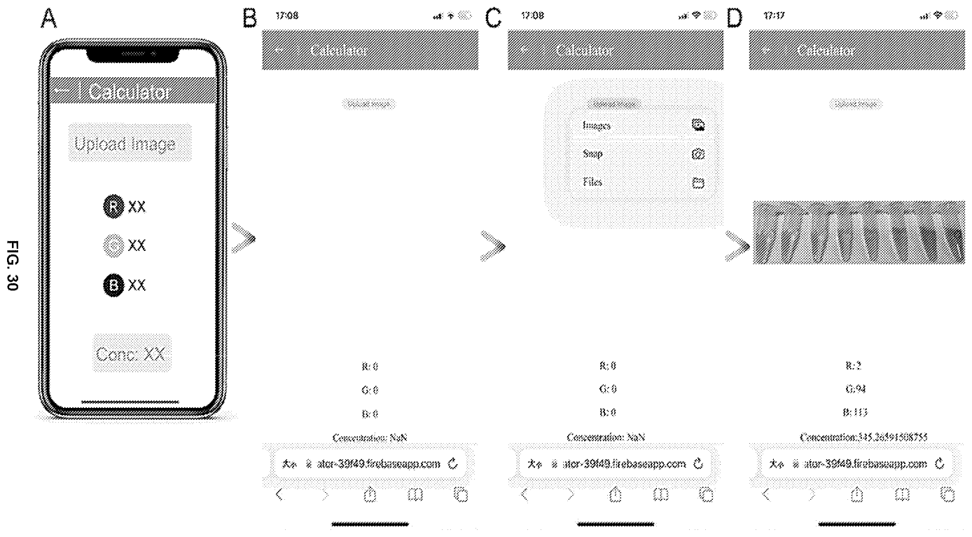

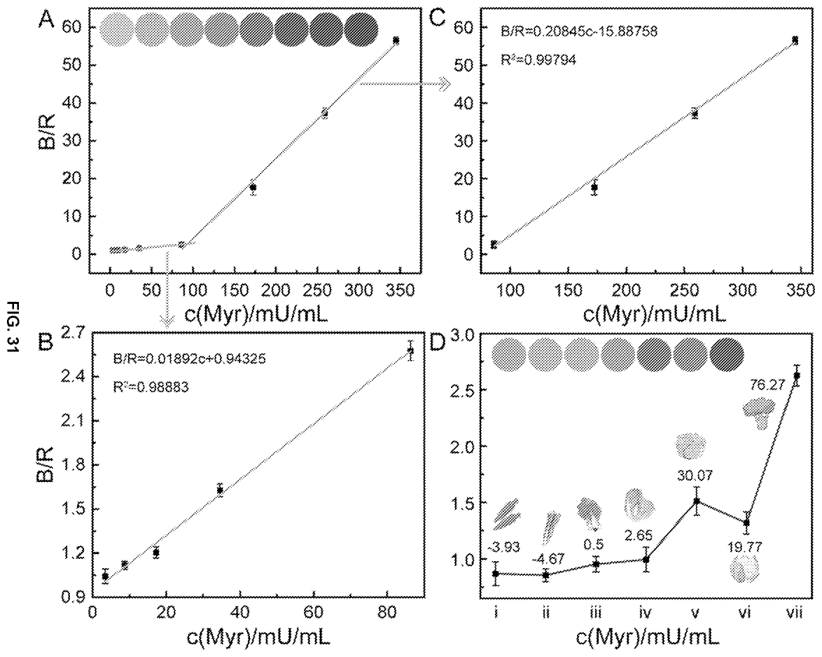

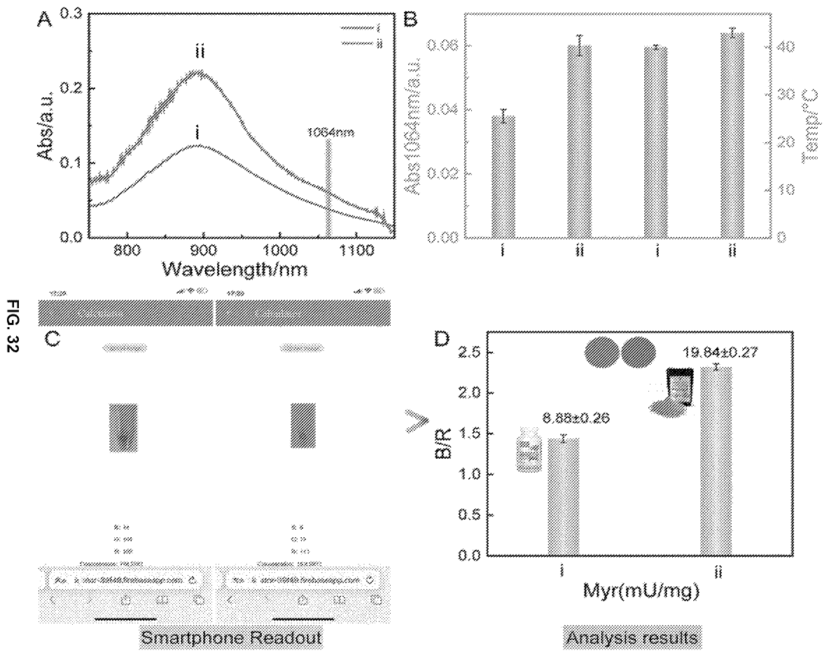

For accurate intelligent onsite Myr analysis, the processing system “Calculator”, which involves a conversion formula, was introduced to on-site examine Myr intelligently. The image analysis and processing flowchart of “Calculator” is illustrated in FIG. 29, and screenshots of “Calculator” are exhibited in FIG. 30. The B/R values were used as a parameter from graphical visualization for quantifying real samples. As depicted in FIG. 29, there is a flow chart 2900 that includes the steps of obtaining a reference image 2910, identifying and decomposing colour to RGB 2920, establishing the relationship between the value of B/R and Myr concentration and fitting the formula 2930, uploading images of actual samples and obtaining RGB 2940, obtaining Myr concentration in actual samples according to the formula 2950, and saving the results 2960. After uploading and decomposing colorimetric images using “Calculator”, the Myr contents in vegetables were obtained via smartphone readout (FIG. 26C). The B/R values and corresponding Myr concentrations are exhibited in FIG. 26D. The highest Myr content was found in broccoli, which measured to be 18.50±1.58 mU/mg, whereas the Myr contents in lettuce and carrot were below zero, indicating that these non-cruciferous vegetables contained no Myr. Similarly, the Myr concentrations calculated from temperatures corresponding to infrared thermal images follow a consistent trend, indicated in Table 2, which further validated the capability of synergistically screening through NIR-II-activating photothermal and colorimetric signals.

The B/R values were used as a parameter for Myr quantification, and FIG. 31A reveals that the ratio of B and R increased with the increment of Myr concentration. From FIG. 31B, the values of B/R displayed a linear relationship with Myr concentration in the range of 0 to 86.25 mU/mL (R²=0.98883) with LOD of 2.48 mU/mL, however, when the B/R values exceeded 2.57692 (Myr concentration was larger than 86.25 mU/mL), the B/R value was proportional to Myr concentration with R²=0.99794 (FIG. 31C). Therefore, the proposed method possesses promising potential for the determination of Myr over a broad range of 0 to 345 mU/mL in two consecutive linear ranges. After uploading and decomposing colorimetric images using “Calculator”, the Myr content in vegetables was obtained via smartphone readout. The B/R value and corresponding Myr concentrations are exhibited in FIG. 31D. The highest Myr content was found in broccoli, which measured 76.27 mU/mL, whereas the Myr content in lettuce and carrot was below zero, indicating that these non-cruciferous vegetables contained no Myr.

In addition, as shown in FIGS. 32A-B, the absorption spectrum and temperature change of the dietary supplement and wasabi powder were also acquired. The results in Table 3 reflected that the Myr amount in wasabi powder is much higher than dietary supplement BroccoMax. Further, the Myr content was determined with the aid of “Calculator”, and according to the results of FIGS. 32C-D, the Myr content in 1 mg BroccoMax is about 8.88±0.26 mU, whereas the Myr concentration in 1 mg of wasabi powder is approximately 19.84±0.27 mU, reaffirming the fact that the amount of Myr in wasabi powder is greater than in BroccoMax. All of these results ascertain that the smartphone-based NIR-II absorption-driven photothermal and colorimetric sensing platform exhibits satisfactory performance for the intelligent, convenient, and interference-free detection of Myr in actual samples.

TABLE 3
Myr amounts in per mg actual samples calculated by
the calibration curves of Abs1064nm and temperature.
The concentration of wasabi powder and dietary supplements
was 1 mg/mL and 10 mg/mL, respectively.

| Real | Abs1064nm | Myr | Temp | Myr |
|---|---|---|---|---|
| Samples | (a.u.) | (mU/mg) | (° C.) | (mU/mg) |
| Dietary | 0.06011 ± 0.003 | 6.9 ± 0.68 | 43.0 ± 1.00 | 5.22 ± 0.75 |
| supple- | | | | |
| ments | | | | |
| Wasabi | 0.03803 ± 0.0021 | 18.65 ± 4.79 | 40.0 ± 0.43 | 19.11 ± 2.52 |
| powder | | | | |

In order to achieve rapid onsite Myr analysis, we have exploited a smartphone-based intelligent processing system named “Calculator” based on the relationship between the B/R values of the obtained colorimetric or thermal images and the Myr concentrations, and successfully applied them for precisely quantitating Myr in real samples including various vegetables, dietary supplements, and commercial wasabi powder. The temperature-responsive and colorimetric sensing platform not only enables rapid qualitative and quantitative Myr onsite analysis via smartphone readout but also shows potential applications in the fields of food quality and nutrition analysis.

As will be appreciated, the method disclosed herein possesses some advantages as listed below:
(i) it is unnecessary to extract and purify proteins, which significantly reduces costs and preparation time for samples;
(ii) the usage of absorbance at 1064 nm could exclude the interference caused by impurities in plant samples, making it more appropriate for the analysis of Myr in whole plant samples;
(iii) temperature signals and infrared thermal images generated by the photothermal effect of CTC could further improve the credibility of detection, which makes the results more reliable; and
(iv) with the assistance of smartphone readout, the colorimetric images could be processed by a website called “Calculator” on smartphone to obtain their RGB values, which are relevant to Myr concentrations, and achieve rapid onsite Myr detection with favorable results according to the relationship functions between B/R values and Myr concentrations. The colorimetric and temperature-responsive sensing platform not only enables rapid qualitative and quantitative Myr onsite analysis via smartphone readout, but also shows potential applications in the fields of food quality analysis.

In summary, the present disclosure discloses a new method for rapid interference-free detection of Myr based on the TMB and H₂O₂ reaction catalyzed by AuNPs and smartphone readout. Generally, H₂O₂ will generate under the catalysis of Myr and GOx, then CTC with blue color and NIR-II absorption would be formed after TMB and AuNPs were added into the above solution containing H₂O₂. Myr could be detected owing to the colorimetric and photothermal effect of CTC. For intelligent onsite Myr analysis accurately, we analyzed the relationship between the B/R values of the colorimetric images and Myr concentrations using a website named “Calculator” on smartphone. The proposed method is able to determine Myr within a wide range of 0 to 345 mU/mL in two consecutive linear ranges. The amounts of Myr in 10 mg/ml of broccoli, green cabbage, cauliflower, Chinese cabbage, Xiao Bai Cai, dietary supplements and wasabi power were about 76.27, 30.07, 19.77, 2.65, 0.5, 18.66 and 88.97 mU/mL, respectively. Hence, it is believed that the colorimetric and temperature-based sensing platform will provide a new technology for the intelligent and precise onsite detection of Myr and open a new horizon for food quality evaluation.

Therefore, the present disclosure provides a new method for rapid and interference-free detection of Myr based on the NIR-II absorption-driven photothermal and colorimetric synergistic sensing system. The method possesses some forte as below:
(i) the usage of NIR-II absorbance at 1064 nm could largely exclude the interference caused by impurities and chlorophyll in plant samples, making it more appropriate for the analysis of Myr in real plant samples;
(ii) temperature signals and near-infrared thermal images generated by the photothermal effect of CTC could further improve the credibility of detection, which makes the results more reliable; and
(iii) with the assistance of smartphone readout, the colorimetric images could be processed by the intelligent processing system “Calculator” to obtain their RGB values, which are relevant to Myr concentrations, and thus achieve simple and rapid onsite Myr detection from complex and dynamic analytes with favorable results.

We believe that such a NIR-II absorption-driven photothermal and colorimetric-based sensing platform will provide a new technology for the intelligent and precise onsite detection of Myr and open a new horizon for food quality assessments and nutritional composition profiles that can benefit the relevant research fields in the future. Overall, the method disclosed herein provides a novel method for onsite Myr analysis in actual foods with the advantages of extraction-free, precise, inexpensive, and intelligent. In addition, we believe it could be applied in the field of food quality evaluations.

### Example 11. Engineering of Gut Bacterial Extracellular Vesicles for Diet-Guided Gastrointestinal Theranostics Through Orthogonal Labelling

Major gastrointestinal diseases including inflammation and cancers, lead to increasing morbidity and mortality worldwide. Gastrointestinal screening and locoregional enrichment and targeted therapeutics are highly required. For example, we can make use of cooperating prestigious enzymatic process, and advantages from human gut microbiota-derived extracellular vesicles (EVs). Further, dietary available gastrointestinal theranostic accumulation may be used in screening and therapy.

EVs are heterogeneous, phospholipid bilayer-enclosed biological particles that regulate cell communication. Gut microbiota-derived EV that retained with parental bacterium functions play critical roles in biological behaviors both interspecifically and intraspecifically. In this example, we isolated and purified EV from gut symbiont Bacteroides thetaiotaomicron carrying bioactive enzyme, conducted enhancement through engineering approaches, and created substrate-based probes bearing tissue affinity, bio-orthogonal, fluorescent, or medicinal functionality. Using the engineered gut bacterial EVs and enzymatic-activated glucosinolate probes, we can achieve the locoregional accumulation of theranostics agents under specific gastrointestinal environment for theranostic mode, including spatio-temporal monitoring of intestinal through NIR-II imaging and sustained therapeutics effect.

The bacterial cultured media or milk whey without caseins were sonicated for an hour before undergoing centrifugation 10000×g for 30 min at 4° C. The supernatants were filtered through polyethersulfone (PES) membranes (0.22 μm pore size) (sartorius) to remove debris and cells. The filtered supernatant was centrifuged for another 30 min at 10,000×g at 4° C. to remove cellular debris. The resultant supernatant was transferred with the pipet to 70 mL Polycarbonate Bottles (Beckman coulter) and centrifuged for 90 min using Rotor Type 45 Ti rotor (Beckman coulter) in Optima XPN Ultracentrifuge (Beckman coulter) at 100,000×g at 4° C. Supernatant were removed completely by using pipets. The obtained crude EVs were suspended in PBS.

A series of sucrose solutions ranging from 10% to 90% were prepared. The crude EVs were resuspended in 90% sucrose and added into the bottle of 13.2-mL ultra-clear tubes (Beckman coulter). Subsequently, sucrose gradient solutions from 80% to 10% were overlayed on top of the crude EVs suspensions. The gradient was ultracentrifuged at 4° C. at 144,000 g for 17 h in SW41 Ti rotor. The fractions were collected carefully from top to bottom after ultracentrifuge and were washed in PBS via ultracentrifuge in the 70 mL Polycarbonate Bottles at 100,000 g for 90 min using Rotor Type 45 Ti rotor. After ultracentrifugation, the supernatant was poured out and the EVs were resuspended in PBS for future use.

Protein concentration was quantified by a bicinchoninic acid (BCA) assay (Thermo Fisher Scientific). A standard curve was plotted using a serial dilutions of bovine serum albumin (BSA). Samples were measured in minimum of three independent replicates and quantified using the standard curve. Total phosphate content was measured using a malachite green phosphate assay (Sigma-Aldrich).

EVs were negative stained and observed using transmission electron microscopy (TEM). The obtained EVs were first mixed with equal volume of 2% PFA. The mixture was deposited on the parafilm to form a droplet. The Formvar-carbon coated EM grids were floated on the droplet for hours in a sterile and dry environment. Subsequently, the Formvar-carbon coated TEM grids were washed in drops of PBS and DI water on a sheet of Parafilm before transferred to 1% glutaraldehyde droplet for 5 min. Finally, the Formvar-carbon coated EM grids were washed in DI water to remove the excess glutaraldehyde. Grids were air-dried before analysis using JEM-1400Flash Electron Microscope and TEM JEOL 2010 HR.

### Results and Discussion

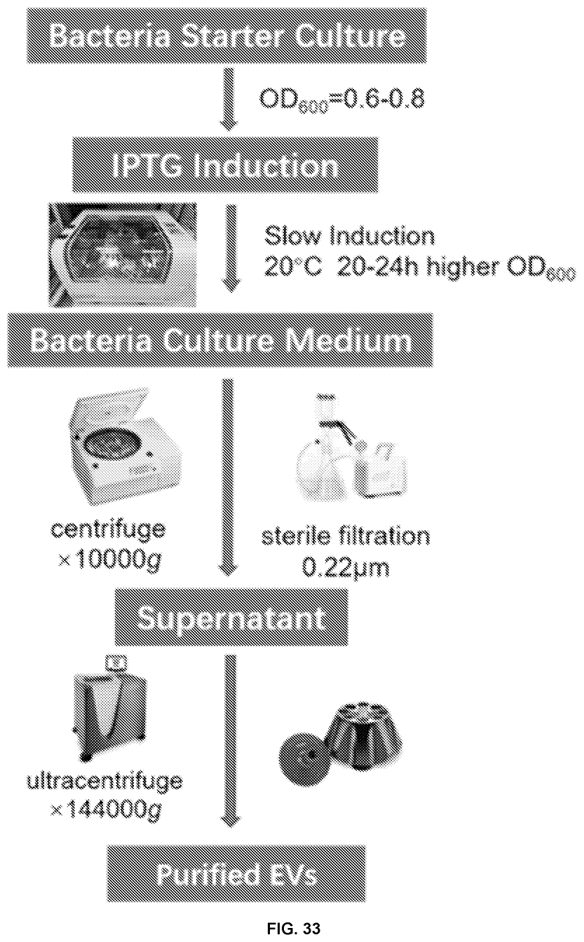

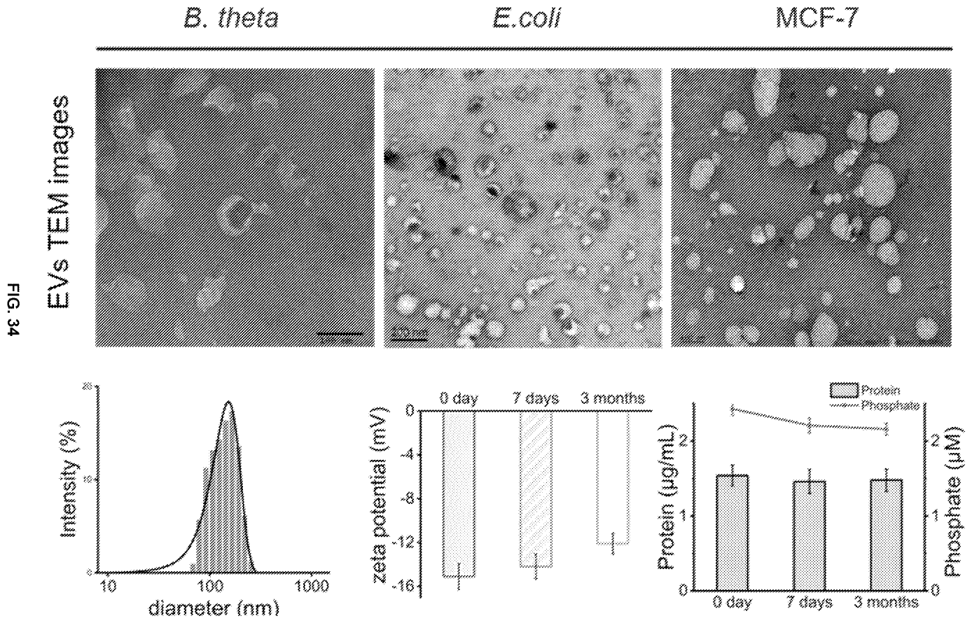

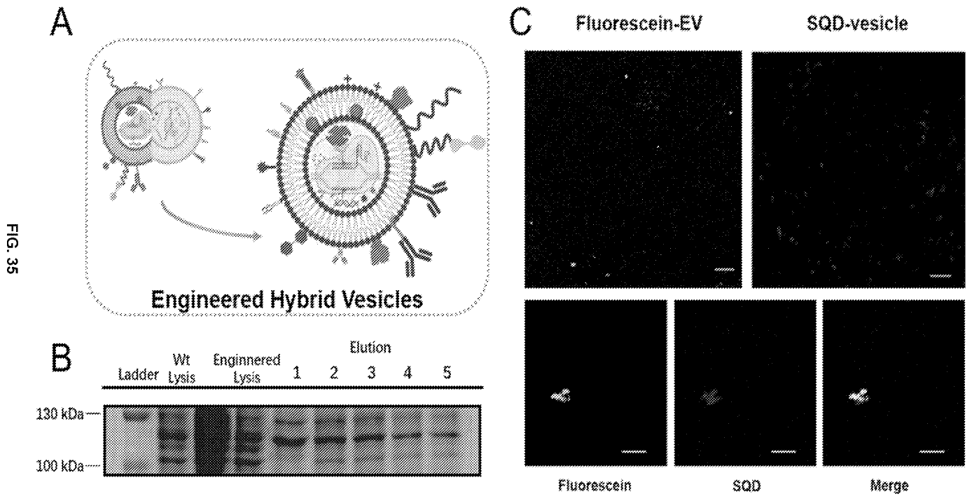

A universal EV extraction method was developed (FIG. 33). FIG. 34 depicts the characterization results of obtained EV from various sources.

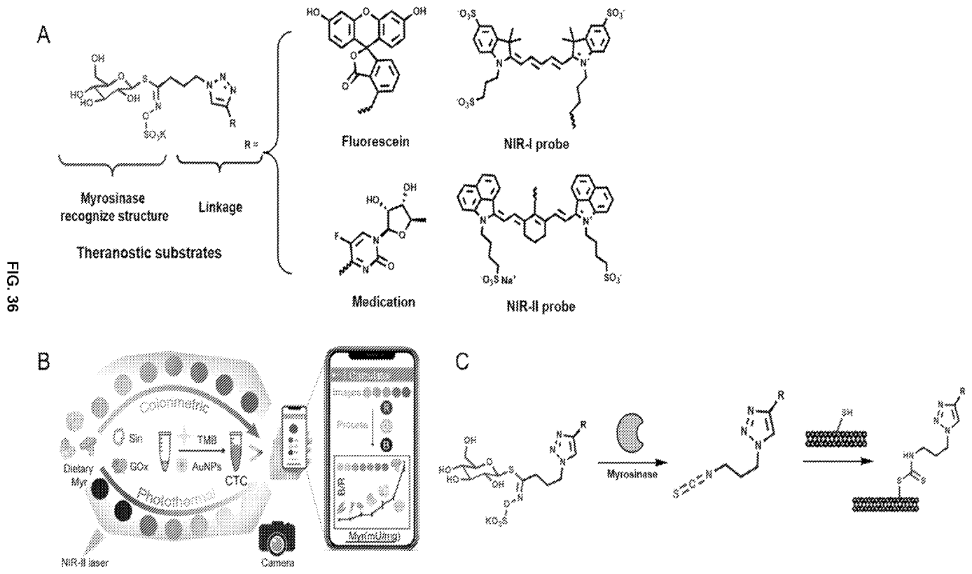

The engineering enhancement of EV is shown in FIG. 35. Hybridization with artificial vesicles provided improved stability and biocompatibility. Gene engineering of parental cells was carried out for improved enzyme expression and activity.

### Example 12. Development of Substrate-Based Versatile Theranostic Agents

For full utilization of myrosinase-glucosinolate based chemistry, a molecular library with various theranostics substrates was established through simple and reliable click chemistry.

Firstly, artificial aglycone with azido functional group was prepared according to a previous article (C. P. Glindemann et al., ChemBioChem 2019, 20, 2341). Briefly, the azido-chlorooxime was synthesized following the aldoxime pathway, followed by the coupling with thiol glucose to yield the thiohydroximates. After the sulfation and deacetylation, the substrate-based azido precursor was obtained. Next, various probes and therapeutic agents including Fluorescein, 5-Fluorouracil, and cyanine probes with NIR-I/II fluorescence, were subsequently linked with aglycone to form artificial glucosinolates.

The as-prepared glucosinolates in-situ conjugate to biological sites after hydrolysis by myrosinase to achieve bioconjugation for myrosinase screening and targeted therapy.

### Results and Discussion

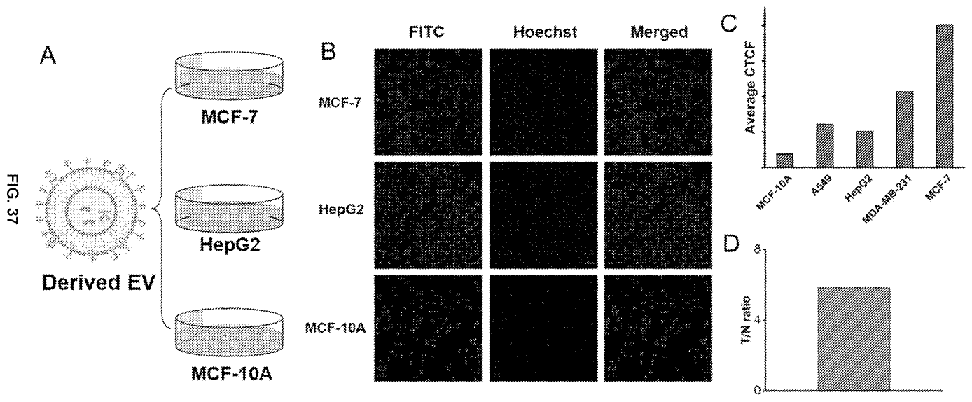

A molecular library of theranostic Myr substrate was developed for novel Myr activity screening technology and targeted therapy through bioconjugation (FIG. 36).

### Example 13. Verification of “Homing Effect” for Lesion Affinity

We planned to verify the homing effect of EV as a bioactive agent carrier for targeted delivery and activation of as prepared theranostics substrates.

Exosome suspension were diluted with PBS and added lipid and membrane marker DiO DMSO stock solution of 2 mg/mL and make it into a final concentration of 20 μg/mL. Exosome was stained for 1 h under room temperature, followed by wash with PBS and collection by 120000 g, 70 min centrifugation for 2 rounds. The stained exosome pellets were resuspended in 200 μL for cell culture.

The MCF-7 cells, MDA-MB-231 cells, Hela cells, HepG2 cells and A549 cells were seeded at 2×10⁵ cells/well in 35-mm diameter μ-dish plastic-bottom (ibidi GmbH, Germany) in complete medium for 24 h at 5% CO₂ and 37° C. The cells were then incubated with 50 μL extracted exosome PBS suspension for 3 h. Subsequently, the excess compound was removed and the cells were washed with PBS 3 times prior to incubation with Hoechst 33258 (2 μg/mL) for 15 min. Excess Hoechst 33258 was removed and cells were washed again for 3 times with PBS and examined under Zeiss LSM 800 confocal microscopy immediately.

### Results and Discussion

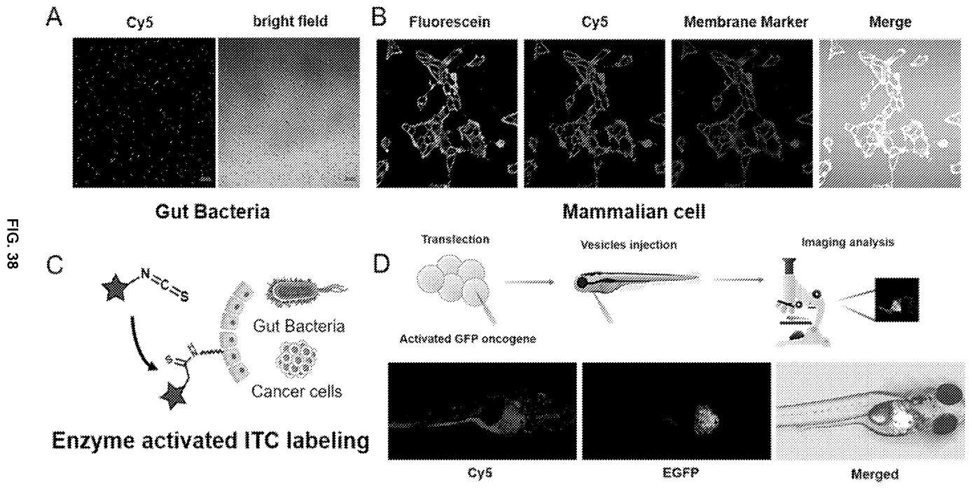

FIG. 37 depicts the results of targeting ability of obtained EV to the parental sites. “Homing effect” of EVs is verified and the results indicate EV as a promising vesicles carrying bioactive compounds including myrosinase to achieve in-situ activation of theranostic substrates.

### Example 14. Imaging and Theranostic Evaluation in Different Levels

The novel theranostic mode following myrosinase enzymatic process was further evaluated in different biological levels including bacteria, mammalian cells and zebra fish model.

Bacteroides thetaiotaomicron (ATCC 29148) and E. coli (ATCC 25922) were incubated in BHIS medium and LB medium at 37° C. until the midexponential phase. GL-Cy5 was then added into the culture mediums to a final concentration of 100 μM. After culturing for 3 h, the bacteria were washed with PBS twice and then suspended in PBS to a final OD₆₀₀=0.25. bacteria solution was dropped on the glass slide and observed by CLSM. The real-time images were taken by CLSM over a continuous time for 10 min. As described above, the 640 nm excitation was used for imaging GL-Cy5 labeling of B. theta and E. coli.

The MCF-7 cells were seeded at 2×10⁵ cells/well in 35-mm diameter μ-dish plastic-bottom (ibidi GmbH, Germany) in complete medium for 24 h at 5% CO₂ and 37° C. The cells were then incubated with 50 μL extracted exosome PBS suspension for 3 h. Subsequently, the excess compound was removed and the cells were washed with PBS 3 times prior to incubation different probes including GL-Cy5 (10 UM), Fluorescein (10 UM) and membrane marker (2 μg/mL) for 15 min. Excess probes was removed and cells were washed again for 3 times with PBS and examined under confocal microscopy immediately.

GFP oncogene transfected zebrafish embryos at 12 hpf were injected with extracted exosome suspension and incubated for 8 hours at 28° C. After that, embryos were incubated with GL-Cy5 at 28° C. for 16 h before visualization of yolk extension phenotypes under a Zeiss LSM 800 confocal microscope using the bright-field channel.

### Results and Discussion

FIG. 38 depicts the results of enzyme activated ITC labeling at different biological levels. The results suggest this myrosinase activated bioconjugation were realized for enzyme screening and targeted therapy.

Therefore, we have disclosed herein a novel theranostic mode inspired by a prestigious enzymatic process by human gut microbiome to achieve locoregional accumulation for monitoring and treatment. Further, the present disclosure provides innovative insights on the potential strategies of utilizing gut microbiome-derived extracellular vesicles for gastrointestinal lesions non-invasive inspection means and targeting therapeutics.

1. A method of quantitatively determining the concentration of active myrosinase in a sample, the method comprising the steps of:
(a) adding a portion of the sample comprising active myrosinase to a first analyte assay formulation for a first period of time to generate an analyte sample, the first analyte assay formulation comprising:
a substrate for active myrosinase that provides glucose as a reaction product;
a glucose oxidase;
either a near infra-red absorbing and photothermally-responsive compound capable of forming a charge transfer complex or nanoparticle aggregates; and
either a peroxidase or nanoparticles having peroxidase-like activity;
(b) adding a portion of the sample comprising inactivated myrosinase to a second analyte assay formulation for a second period of time to generate a analyte reference sample, the second analyte assay formulation being the same as the first analyte assay formulation and the second period of time being the same as the first period of time;
(c) determining the concentration of the active myrosinase in the sample by measuring one or more of:
(i) Red Green Blue colour values in the analyte sample and Red Green Blue colour values in the analyte reference sample, calculating the Red/Blue ratio for each of the analyte sample and the analyte reference sample and calculating the difference between the Red/Blue ratios to provide a sample Red/Blue ratio and comparing the sample Red/Blue ratio obtained to a pre-determined calibration curve of active myrosinase concentration based on Red/Blue ratios obtained from known concentrations of active myrosinase;
(ii) an infra-red image of the analyte sample and an infra-red image of the analyte reference sample obtained following irradiation of the analyte sample and the analyte reference same by a laser for a third period of time and calculating the temperature difference between a temperature obtained from the infra-red image of the analyte sample and a temperature obtained from the infra-red image of the analyte reference sample and comparing the difference value obtained to a pre-determined calibration curve of active myrosinase concentration based on temperatures obtained from infra-red images of known concentrations of active myrosinase;
(iii) an absorbance spectra of the analyte sample and an absorbance spectra of the analyte reference sample and calculating the difference between the absorbance spectra of the analyte sample and the absorbance spectra of the analyte reference sample and comparing the difference value obtained to a pre-determined calibration curve of active myrosinase concentration based on absorbance spectra of known concentrations of active myrosinase; and
(iv) a temperature signal obtained following irradiation of the analyte sample and the analyte reference sample by a laser for a third period of time and calculating the difference between the temperature signal of the analyte sample and the temperature signal of the analyte reference sample and comparing the difference value obtained to a pre-determined calibration curve of active myrosinase concentration based on temperature signals of known concentrations of active myrosinase.

2. The method according to claim 1, wherein two or more of (i) to (iv) are used to determine the concentration of active myrosinase in the sample.

3. The method according to claim 1, wherein the substrate for active myrosinase that provides glucose as a reaction product is a glucosinolate.

4. The method according to claim 1, wherein the near infra-red absorbing and photothermally-responsive compound capable of forming a charge transfer complex or nanoparticle aggregates is selected from phenylboronate decorated gold nanoparticles, or one or more of the group selected from perylene, F4TCNQ, tetracyanoquinodimethane (TCNQ), and 3,3′,5,5′-tetramethylbenzidine.

5. The method according to claim 1, wherein the nanoparticles having peroxidase-like activity are selected from silver or, more particularly, gold nanoparticles.

6. The method according to claim 1, wherein one or more of the following apply:
(bi) when the nanoparticles having peroxidase-like activity or the peroxidase are gold nanoparticles, they are present in the first and second analyte assay formulations at a concentration of from 0.02 to 0.8 nM;
(bii) when the near infra-red absorbing and photothermally-responsive compound capable of forming a charge transfer complex or nanoparticle aggregates is 3,3′,5,5′-tetramethylbenzidine, then it is present in the first and second analyte assay formulations at a concentration of from 0.5 to 3 mM;
(biii) the glucose oxidase is present in an amount of from 5 U/mL to 25 U/mL;
(biv) the substrate for active myrosinase that provides glucose as a reaction product is present in an amount of from 0.05 mM to 0.75 mM.

7. The method according to claim 1, wherein the first and second analyte assay formulations further comprise an acetate buffer in an amount that provides a pH of from 3.5 to 7.5.

8. The method according to claim 1, wherein the sample comprising active myrosinase and the sample comprising inactivated myrosinase is provided at a concentration of from 1 to 20 mg/mL.

9. The method according to claim 1, wherein the sample comprising inactivated myrosinase is obtained by heating a sample comprising active myrosinase to a temperature suitable to denature active myrosinase for a fourth period of time.

10. The method according to claim 1, wherein the sample comprising active myrosinase is subjected to incubation for a fifth period of time at a suitable temperature before use in the method.

11. The method according to any claim 1, wherein when one or both of (ii) and (iv) of step (c) in claim 1 are used to determine the concentration of the active myrosinase in the sample, then the laser is a near infra-red light laser.

12. The method according to claim 1, wherein the first and second periods of time are from 10 minutes to 1 hour.

13. The method according to claim 1, wherein the absorbance spectra of the analyte sample and the analyte reference sample are based on their absorbance at a specified wavelength or a wavelength range in the near infra-red range.

14. The method according to claim 1, wherein the sample is obtained from a dietary supplement comprising myrosinase, wasabi (e.g. wasabi powder), or a cruciferous plant.

15. The method according to claim 1, wherein the first and second analyte assay formulations comprise:
sinigrin;
a glucose oxidase;
3,3′,5,5′-tetramethylbenzidine; and
gold nanoparticles.

16. The method according to a claim 1, wherein the method further comprises a step (d), which is selected from one or more of the following:
(di) retaining or disposing of a batch from which the sample was drawn based on a level of freshness of the batch based on the concentration of the active myrosinase in the sample; and
(dii) labelling a batch from which the sample was drawn with a specific level or amount of active myrosinase.

17. A formulation for use in a method of quantitatively determining the concentration of active myrosinase in a sample, the formulation comprising:
a substrate for active myrosinase that provides glucose as a reaction product;
a glucose oxidase;
either a near infra-red absorbing and photothermally-responsive compound capable of forming a charge transfer complex or nanoparticle aggregates; and
either a peroxidase or nanoparticles having peroxidase-like activity.

18. The formulation according to claim 17, wherein the substrate for active myrosinase that provides glucose as a reaction product is a glucosinolate.

19. The formulation according to claim 17, wherein the near infra-red absorbing and photothermally-responsive compound capable of forming a charge transfer complex or nanoparticle aggregates is selected from phenylboronate decorated gold nanoparticles, or one or more of the group selected from perylene, F4TCNQ, tetracyanoquinodimethane (TCNQ), and 3,3′,5,5′-tetramethylbenzidine.

20.-21. (canceled)

22. A kit of parts suitable to provide a formulation according to claim 17, wherein the kit of parts comprises:
a substrate for active myrosinase that provides glucose as a reaction product;
a glucose oxidase;
either a near infra-red absorbing and photothermally-responsive compound capable of forming a charge transfer complex or nanoparticle aggregates; and
either a peroxidase or nanoparticles having peroxidase-like activity.

23.-24. (canceled)
